(19) United States
(12) Patent Application Publication
(10) Pub. No.: US 2022/0387392 A1
(43) Pub. Date: Dec. 8, 2022

(54) METHODS AND COMPOSITIONS RELATED TO THE USE OF ERGOTHIONEINE

(71) Applicants: Steven CHEN, Coto De Caza, CA (US); Priscilla SAMUEL, Algonquin, IL (US); Elsa Cecilia COLLUM, Waxhaw, NC (US); BLUE CALIFORNIA, Rancho Santa Margarita, CA (US)

(72) Inventors: Steven CHEN, Coto De Caza, CA (US); Priscilla SAMUEL, Algonquin, IL (US); Elsa Cecilia COLLUM, Waxhaw, NC (US)

(21) Appl. No.: 17/760,056

(22) PCT Filed: Feb. 3, 2021
(86) PCT No.: PCT/US21/16320

§ 371 Date: Aug. 3, 2022

Related U.S. Application Data
(60) Provisional application No. 62/969,645, filed on Feb. 3, 2020.
(60) Provisional application No. 62/969,607, filed on Feb. 3, 2020.
(60) Provisional application No. 63/051,211, filed on Jul. 13, 2020.

(51) Int. Cl. A61K 31/4172; A61P 25/28; A61P 39/06; A61P 31/14; A01N 1/02

(52) U.S. Cl. CPC A61K 31/4172; A61P 25/28; A61P 39/06; A61P 31/14; A01N 1/0226

(57) ABSTRACT

Provided herein are compositions comprising ergothioneine (e.g., a natural form of purified L-ergothioneine) and methods of using such compositions, e.g., for improving cell viability, maintaining telomere length, or treating telomere-related disorders. In some embodiments, the ergothioneine has a purity of at least or about 98%.

### RELATED APPLICATIONS

This application claims the benefit of the filing date of U.S. Provisional Application No. 62/969,607, entitled “METHODS AND COMPOSITIONS RELATED TO THE USE OF ERGOTHIONEINE”, filed Feb. 3, 2020; U.S. Provisional Application No. 62/969,645, entitled “METHODS AND COMPOSITIONS RELATED TO THE USE OF ERGOTHIONEINE”, filed Feb. 3, 2020; U.S. Provisional Application No. 63/051,211, entitled “METHODS AND COMPOSITIONS RELATED TO THE USE OF ERGOTHIONEINE”, filed Jul. 13, 2020; the contents of each of which are incorporated herein by reference in their entirety.

### SUMMARY OF THE INVENTION

Ergothioneine is a naturally occurring metabolite of histidine with a thiol group attached to the C2 atom of the imidazole ring. It is synthesized by bacteria and fungi with antioxidant properties and anti-inflammatory properties and can help to prevent and combat oxidative stress, which can trigger the inflammatory response. The present disclosure is based, at least in part, on the surprising discovery of the ability of ergothioneine to maintain the length of telomeres in cells and the improvement of cell viability. Accordingly, herein are ergothioneine compositions that can be used for such purposes as well as for treating, preventing or reducing the risk of diseases or disorders associated with telomere shortening, preventing or delaying the ageing processes, treating, preventing or reducing the risk of cognition-related diseases or disorders, etc. The compositions and methods provided herein can also be used to treat or prevent oxidative stress-related disorders, diseases or conditions associated with telomere shortening. Methods and compositions for treating, preventing or reducing the risk of oxidative stress-related disorders, diseases or conditions are also provided. Methods of achieving the foregoing results are also provided.

Some aspects of the present disclosure provide methods of improving cell viability by contacting cells with a composition of ergothioneine.

Some aspects of the present disclosure provide methods of maintaining the length of telomeres in cells or inhibiting or preventing telomere shortening in cells by contacting cells with a composition of ergothioneine.

Some aspects of the present disclosure provide methods of reducing the rate of telomere shortening in cells by contacting cells with a composition of ergothioneine.

Some aspects of the present disclosure provide methods of lowering the amount, such as percent, of short telomeres in cells by contacting cells with a composition of ergothioneine.

Some aspects of the present disclosure provide methods of lengthening telomeres in cells by contacting cells with a composition of ergothioneine.

Some aspects of the disclosure provide methods of treating, preventing or reducing the risk of telomere-related disorders, diseases or other conditions. In some embodiments, the method is a method of treating, preventing or reducing the risk of premature ageing in a subject.

Some aspects of the disclosure provide methods of treating, preventing or reducing the risk of a cognition-related disorder, disease, or other condition.

Some aspects of the disclosure provide methods of treating, preventing or reducing the risk of an oxidative stress-related disorder, disease, or other condition. In some embodiments, the oxidative stress-related disorder, disease, or other condition is a viral infection (e.g., a respiratory viral infection). In some embodiments, the oxidative stress-related disorder, disease, or other condition is COVID-19.

In some embodiments of any one of the methods or compositions provided herein, the contacting is in vitro. In some embodiments, the contacting is by incubating cells, such as a test cell population, with a composition of ergothioneine.

In some embodiments of any one of the methods or compositions provided herein, the contacting is in vivo. In some embodiments, the contacting is by administering ergothioneine such that the ergotheioneine comes in contact with cells in a subject.

In some embodiments of any one of the methods or compositions provided herein, the composition comprises purified ergothioneine, such as a natural form of purified ergothioneine. In some embodiments of any one of the methods or compositions provided herein, the purified ergothioneine has purity of about 98% or is at least 98%.

In some embodiments of any one of the methods or compositions provided herein, the ergothioneine is L-ergothioneine.

In some embodiments of any one of the methods or compositions provided herein, the composition is in a free-flowing powder form.

In some embodiments of any one of the methods or compositions provided herein, the ergothioneine contacted with the cells, such as incubated, such as in a test cell population, is in the amount of about 0.04 mg/ml to about 1 mg/ml.

In some embodiments of any one of the methods or compositions provided herein, the ergothioneine contacted with the cells, such as incubated, such as in a test cell population, is in the amount of about 0.04 mg/ml or at least 0.04 mg/ml. In some embodiments of any one of the methods or compositions provided herein, the ergothioneine contacted with the cells, such as incubated, such as in a test cell population, is in the amount of about 0.1 mg/ml or at least 0.1 mg/ml. In some embodiments of any one of the methods or compositions provided herein, the ergothioneine contacted with the cells, such as incubated, such as in a test cell population, is in the amount of about 0.3 mg/ml or at least 0.3 mg/ml. In some embodiments of any one of the methods or compositions provided herein, the ergothioneine contacted with the cells, such as incubated, such as in a test cell population, is in the amount of about 1 mg/ml or at least 1 mg/ml.

In some embodiments of any one of the methods or compositions provided herein, the cell viability is accomplished by or measured by maintaining the average telomere length of the cells, such as of a test cell population, or preventing or inhibiting telomere length shortening in the cells, or slowing the rate of telomere length shortening in the cells, or lowering the amount, such as percent, of short telomeres in the cells, or lengthening the telomeres in the cells.

In some embodiments of any one of the methods or compositions provided herein, the average telomere length of cells, such as of a test cell population, is maintained due to contact, such as by incubation or administration, with the ergothioneine composition as compared with the average telomere length of control cells, such as a control cell population, in the absence of such contact. In some embodiments of any one of the methods or compositions provided herein, the average telomere length of cells, such as of a test cell population, due to contact, such as by incubation or administration, with the ergothioneine composition is equal to or greater than the average telomere length of control cells, such as of a control cell population, in the absence of such contact. In some embodiments of any one of the methods or compositions provided herein, telomere shortening is slowed in cells, such as of a test cell population, due to contact, such as by incubation or administration, with the ergothioneine composition as shown by the average telomere shortening rate of the cells being less than the average telomere shortening rate of control cells, such as of a control cell population, in the absence of such contact. In some embodiments of any one of the methods or compositions provided herein, the amount, such as percent, of short telomeres in the cells is lowered. In some embodiments of any one of the methods or compositions provided herein, the telomeres of the cells are lengthened.

In some embodiments of any one of the methods or compositions provided herein, the telomere length is determined by a single cell telomeric mapping technique. In some embodiments of any one of the methods or compositions provided herein, the single cell telomeric mapping technique is based on a fluorescent in situ hybridization assay.

In some embodiments of any one of the methods or compositions provided herein, the cells, such as the test cell population, are skin cells or are from a tissue sample from the skin. In some embodiments of any one of the methods or compositions provided herein, the cells, such as the test cell population, are intestinal cells or are from a tissue sample from the small intestine. In some embodiments of any one of the methods or compositions provided herein, the cells, such as the test cell population, are testicular cells or are from a tissue sample from the testis. In some embodiments of any one of the methods or compositions provided herein, the cells, such as the test cell population, are corneal cells or are from a tissue sample from the cornea. In some embodiments of any one of the methods or compositions provided herein, the cells, such as the test cell population, are neural or brain cells or are from a tissue sample from the brain. In some embodiments of any one of the methods or compositions provided herein, the cells, such as the test cell population, are blood cells. In some embodiments of any one of the methods or compositions provided herein, the cells, such as the test cell population, are bone marrow cells or are from a bone marrow sample. In some embodiments of any one of the methods or compositions provided herein, the cells, such as the test cell population, are tumor or cancer cells or are from a tissue sample from cancerous or tumor tissues.

In some embodiments of any one of the methods or compositions provided herein, the cells or cell population are from or is a transplantable organ or body part, including tissue, such as heart, lung, heart-lung, kidney, liver, pancreas, intestine, skin, face, hands, leg, penis, bone, uterus, thymus, islets of Langerhans, heart valve, or ovary(ies). In some embodiments of any one of the methods or compositions provided herein, the cells or cell population are from or is a severed limb or other body part, including tissue.

In some embodiments of any one of the methods or compositions provided herein, the cells, such as the control cells, are cells of an immortalized cell line. In some embodiments of any one of the methods or compositions provided herein, the cells, such as the control cells, are Hela 2, HeLa, MCF7, HeLa S3, 293T, L5178Y-S, MEFs BL6 G3 Terc−/−, MEFs BL6 wild type, HeLa 1211, MEFs 129Sv/BL6 wild-type and L5178Y-R cells or are the same type of cells as the test cells or are derived from a tissue sample, such as of the same type, as the test cells.

In some embodiments of any one of the methods provided herein, the methods comprise administering a therapeutically effective amount of composition comprising ergothioniene to a subject in need thereof. In some embodiments of any one of the methods provided herein, the methods comprise administering ergothioneine at a dose of about 5 mg to about 30 mg per day. In some embodiments of any one of the methods provided herein, the methods comprise administering ergothioneine at a dose of about 5 mg to about 25 mg per day. In some embodiments of any one of the methods provided herein, the methods comprise administering ergothioneine at a dose of about 5 mg per day or at least 5 mg per day. In some embodiments of any one of the methods provided herein, the methods comprise administering ergothioneine at a dose of about 25 mg per day or at least 25 mg per day.

In some embodiments of any one of the methods provided herein, the treating, preventing or reducing the risk of diseases or disorders associated with telomere shortening comprises maintaining the average telomere length of cells, preventing or inhibiting the telomere length shortening in the cells, reducing the rate of telomere shortening of cells in a target environment, lowering the amount, such as percent, of short telomeres, or lengthening the telomeres in the subject in need thereof. In some embodiments of any one of the methods provided herein, the diseases or disorders associated with telomere shortening is or is associated with premature ageing or is a cognition-related disorder, disease, or other condition. In some embodiments of any one of the methods provided herein, the diseases or disorders associated with telomere shortening is or is associated with chronic inflammation. In some embodiments of any one of the methods provided herein, the diseases or disorders associated with telomere shortening are caused by or associated with oxidative stress.

In some embodiments of any one of the methods provided herein, the method is for maintaining health, a healthy condition, a healthy body, healthy cells that, for example, can in some embodiments slow the ageing process, reduce the risk of disease, etc.

Some aspects of the disclosure provide compositions, such as pharmaceutical formulations, comprising a therapeutically effective amount of an ergothioneine compositions and, optionally, one or more pharmaceutically acceptable carriers. In one embodiment, any one of the compositions provided herein can be for any one of the purposes provided herein.

### BRIEF DESCRIPTION OF THE DRAWINGS

FIG. 1 shows a diagram of metabolism of ergothioneine and its antioxidant mechanisms. The diagram is adapted from Kerley et al., Free Radical Biology and Medicine, DOI: 10.1016/j.freeradbiomed.2017.12.030.

FIG. 2 shows pathways toward ergothioneine biosynthesis in bacteria and fungi. The diagram is adapted from van der Hoek et al., Front. Bioeng. Biotechnol (2019).

FIGS. 3A and 3B show growth curves of primary human fibroblasts treated with ergothioneine. Cells were cultured under (A) standard conditions or (B) oxidative conditions (10 μM H2O2). Each point on the population curve represents the average of the triplicates for each cell passage. PD: population doubling.

FIG. 4 shows the effect of ergothioneine on relative telomerase activity. Data are mean±standard deviation (n=3). * p<0.05 compared to control.

FIGS. 5A-5D shows the effect of ergothioneine on telomere length under standard conditions. (A) Median telomere length (50th percentile), (B) 20th percentile telomere length, (C) Percent of short telomeres (<3 kbp), (D) Telomere shortening rate. Median telomere length (initial−final)/population doubling. Data are mean±standard deviation (n=3). * p<0.05, ** p<0.01, *** p<0.001 compared to control. Kbp: kilobase pairs; PD: population doubling.

FIG. 6A-6D show the effect of ergothioneine on telomere length under oxidative conditions. (A) Median telomere length (50th percentile), (B) 20th percentile telomere length, (C) Percent of short telomeres (<3 kbp), (D) Telomere shortening rate. Median telomere length (initial−final)/population doubling. Data are mean±standard deviation (n=3). * p<0.05, ** p<0.01, *** p<0.001 compared to control. Kbp: kilobase pairs; PD: population doubling.

FIG. 7A-7D show effects of ergothioneine, including telomere lengthening (FIG. 7D, dose of 0.3 mg/ml).

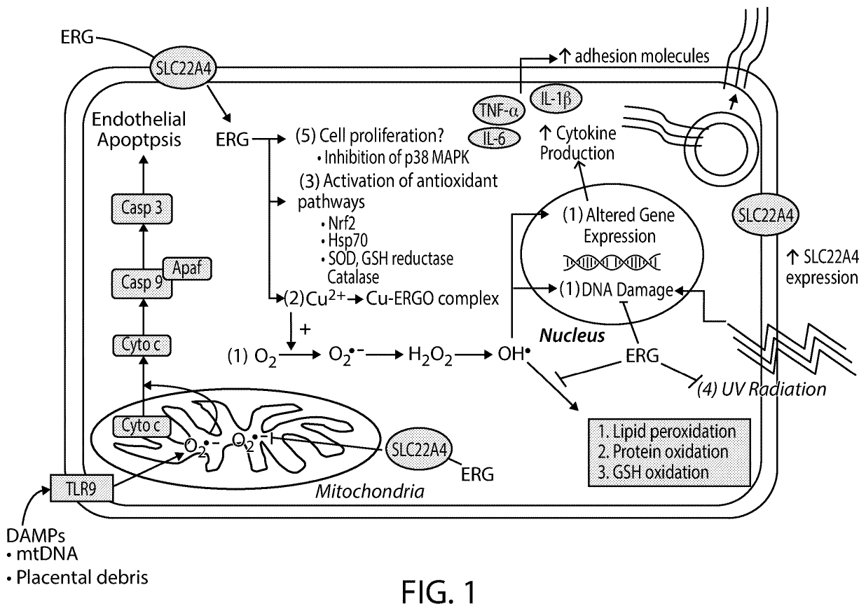

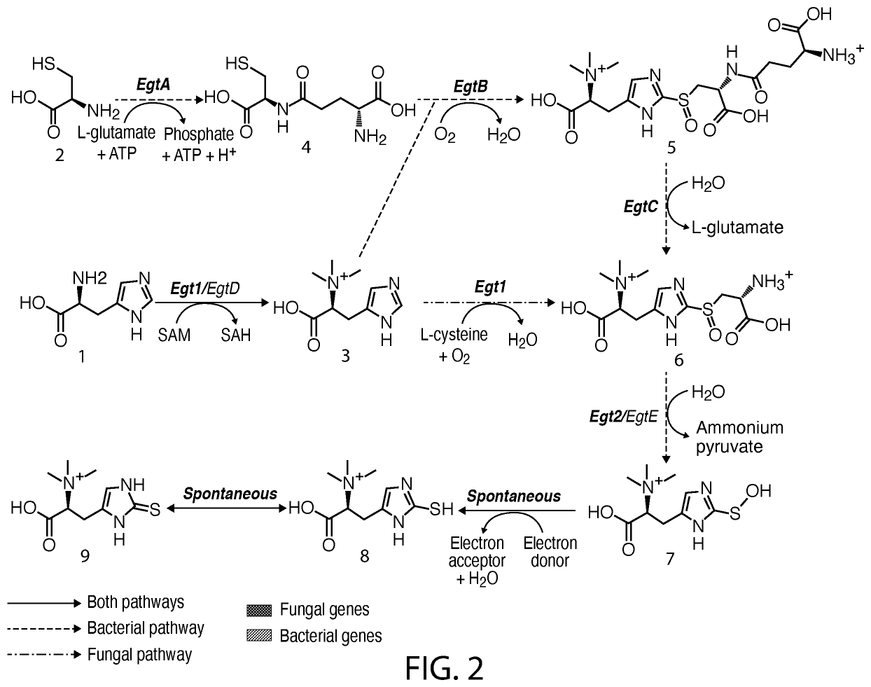

### DETAILED DESCRIPTION

Aspects of the present disclosure relate to the use of ergothioneine to improve in vitro or in vivo cell viability and/or by maintaining the telomere lengths of cells when compared with cells that are not contacted or treated with ergothioneine. Accordingly, methods and compositions, such as pharmaceutical formulations, described herein are useful, in some embodiments, for treating, preventing or reducing the risk of telomere-related disorders, diseases or other related conditions.

The terms “improve,” “improving,” and “improvement” as used herein refers to a process or an action that results in enhanced results when compared with a corresponding counterpart that is not affected by said process or action. As used herein, the terms also refer to a process or an action that results in no change of results when compared with a corresponding counterpart that is not affected by said process or action.

As used herein, “purify” or “purified” refer to freeing something of extraneous contaminating or debasing matter. As used herein, a “substantially pure” preparation of something refers to a preparation having purity of the desired thing of at least 90% (i.e., 90% or greater than 90%). Any one of the ergothioneine compositions provided herein may be substantially pure.

The terms “incubating” and “incubation” as used herein refers to a process of mixing two or more chemical or biological entities (such as a chemical compound and a cell population) and allowing them to interact under conditions favorable for cell culture conditions as disclosed herein.

As used herein, “cell viability” refers to a measure or determination of the proportion of live, healthy cells within a cell population, which can be measured by assays known or understood by a skilled person in the art. Cell viability assays can be used to determine the overall health of cells, optimize culture or experimental conditions, and to measure cell survival following treatment with compounds. For example, as described herein, cell viability can be determined by measuring the average telomere length of cells of interest after any one of the ergothioneine compositions provided herein.

The term “free-flowing powder” or “free-flowing powder form” refers to powder compositions that have satisfying flowability, which is the ability of a powder to flow. For instance, a free-flowing powder may be pourable or may be easy to pour.

Generally, the term “about,” as used herein when referring to a measurable value such as an amount of weight, time, dose, etc. is meant to encompass in one example variations of ±15% or ±10%, in another example ±5%, in another example ±1%, and in yet another example ±0.1% from the specified amount, as such variations are appropriate to perform the disclosed method and for the disclosed compositions, such as pharmaceutical compositions.

It is to be understood that the terminology used herein is for the purpose of describing particular embodiments only and is not intended to be limiting.

### Purified Ergothioneine Compositions

Aspects of the disclosure relate to using purified ergothioneine compositions such as to improve cell viability of cells, such as of a test cell population, or for use in the methods of treating, preventing or reducing the risk of provided herein.

Ergothioneine is a naturally occurring metabolite of histidine with a thiol group attached to the C2 atom of the imidazole ring, which is synthesized by bacteria and fungi with antioxidant properties. Ergothioneine can scavenge oxidizing species that are not free radicals. It has a role as a fungal metabolite, a plant metabolite, a xenobiotic metabolite and a chelator.

Ergothioneine is found ubiquitously in plants and animals and is present in many human foodstuffs. Actinobacteria such as Mycobacterium smegmatis and filamentous fungi such as Neurospora crassa can produce ergothioneine. L-ergothioneine can be found in very small quantities in mushrooms, oats, garlic, animal organs (liver, kidney), and some varieties of beans. Without wishing to be bound by theory, ergothioneine is biosynthesized by fungi and mycobacteria and is captured by plants through their roots. In general, the highest levels of ergothioneine have been found in mushrooms (0.1-1 mg/g dried material).

Animals and plants cannot produce ergothioneine endogenously and therefore, must obtain it from dietary sources or in the case of plants, from their environment. Ergothioneine is rapidly cleared from the circulation and then avidly retained with minimal metabolism. For example, the whole-body half-life of ingested ergothioneine in rats is 1 month. The content of ergothioneine varies greatly among tissues and is strongly dependent on its dietary level. In addition to erythrocytes and bone marrow, high ergothioneine levels have also been found in seminal fluid, eye lens, and skin.

Ergothioneine is a powerful scavenger of hydroxyl radicals and an inhibitor of iron or copper ion-dependent generation of hydroxyl radicals from hydrogen peroxide (H₂O₂). A specific ergothioneine transporter has been identified (gene symbol SLC22A4-PMID 15795384). SLC22A4 is highly expressed in the kidney, where it is thought to aid in active secretion of organic cations, and may facilitate the active reabsorption of ergothioneine. Humans also produce a highly specific transport protein called OCTN1 for L-ergothioneine that makes it highly bioavailable and avidly retained. In addition, ergothioneine appears to play a pivotal protective role in monocytes, because the occurrence of rheumatoid arthritis and Crohn's disease has very recently been linked to variant ergothioneine transporter genes (PMID: 15795384).

Ergothioneine is synthesized from one molecule of L-histidine (1), one molecule of L-cysteine (2), and 3 methyl groups donated by S-adenosyl-L-methionine. The biosynthesis of ergothioneine in bacteria is described in, and incorporated by reference from Richard-Greenblattet et al. (2015) J Bio Chem 290 (38): 23064-23076. The biosynthesis of ergothioneine at least involves: (1) EgtB (or iron(II)-dependent oxidoreductase EgtB), which catalyzes the oxidative sulfurization of hercynine via the addition of oxygen and gamma-glutamyl-cysteine on hercynine (N-alpha,N-alpha,N-alpha-trimethyl-L-histidine); (2) EgtC (or Amidohydrolase EgtC) catalyzes the hydrolysis of the gamma-glutamyl amide bond from N-(gamma-glutamyl)-[N (alpha),(alpha),(alpha)-trimethy 1-L-histidinyl]-cysteine sulfoxide to produce hercynylcysteine sulfoxide; (3) EgtD (or histidine-specific methyltransferase EgtD) catalyzes the methylations of histidine to form N-alpha,N-alpha,N-alpha-trimethyl-L-histidine (also known as hercynine); and (4) EgtE (or pyridoxal-phosphate-dependent protein EgtE) is believed to catalyze the removing of pyruvate, ammonia and oxygen to produce ergothioneine.

In some embodiments, the microbial ergothioneine biosynthesis can be conducted in vitro using an engineered host cell. In some embodiments, suitable host cells can be, for example, bacterial cells and yeast cells. In some embodiments, suitable yeast cells can be Saccharomyces, preferably Saccharomyces cerevisiae. In some embodiments, suitable yeast cells can be Pichia, preferably Pichia pastoris. In some embodiments, the microbial ergothioneine biosynthesis can involve fermentation processes. Methods of producing ergothioneine are described in, and incorporated by reference from PCT/US2015/027977.

The ergothioneine composition can be purified and in a natural form. In some embodiments of any one of the methods or compositions provided herein, a natural form can be derived from bacteria. In some embodiments of any one of the methods or compositions provided herein, a natural form can be derived from plants. In some embodiments of any one of the methods or compositions provided herein, a natural form can be derived from animals. In some embodiments of any one of the methods or compositions provided herein, a natural form can be derived from sources that include but are not limited to liver, kidney, black beans, kidney bean, oat bran, bolete and oyster mushrooms. In some embodiments of any one of the methods or compositions provided herein, a natural form can be derived from any sources that are suitable for the production of a composition as described herein.

In some embodiments of any one of the methods or compositions provided herein, the ergothioneine can be derived from an in vitro bioengineered cell host.

In some embodiments of any one of the methods or compositions provided herein, the form of ergothioneine used in any one of the methods or compositions provided herein is ErgoActive® (Blue California, Rancho Santa Margarita, Calif.). In some embodiments of any one of the methods or compositions provided herein, the ergothioneine comprises L-ergothioneine. In some embodiments of any one of the methods or compositions provided herein, the ergothioneine comprises ergothioneine in any configurations that are suitable for the methods and compositions as described herein. For example, a suitable configuration of ergothioneine is one that improves cell viability and/or maintains telomere length.

In some embodiments of any one of the methods or compositions provided herein, the ergothioneine in the composition as described herein is substantially purified. In some embodiments of any one of the methods or compositions provided herein, the ergothioneine in the composition has purity greater than about 70%, greater than about 71%, greater than about 72%, greater than about 73%, greater than about 74%, greater than about 75%, greater than about 76%, greater than about 77%, greater than about 78%, greater than about 79%, greater than about 80%, greater than about 81%, greater than about 82%, greater than about 83%, greater than about 84%, greater than about 85%, greater than about 86%, greater than about 87%, greater than about 88%, greater than about 89%, greater than about 90%, greater than about 91%, greater than about 92%, greater than about 93%, greater than about 94%, greater than about 95%, greater than about 96%, greater than about 97%, greater than about 98%, or greater than about 99%, inclusive of all ranges and subranges therebetween. In some embodiments of any one of the methods or compositions provided herein, the ergothioneine in the composition as described herein has purity of 98%, 99% or more.

In some embodiments of any one of the methods or compositions provided herein, the ergothioneine composition can be in a free-flowing powder form. Without wishing to be bound by theory, the flow of powder can be affected by factors such as shape of the particles, surface of the particles, and the presence of electrostatic charges. In some embodiments of any one of the methods or compositions provided herein, to improve the flowability, flow enhancers may be used in the composition to reduce interparticle friction. In some embodiments of any one of the methods or compositions provided herein, the ergothioneine composition can be in any form that is suitable for the methods or compositions as described herein.

In some embodiments of any one of the methods or compositions provided herein, the ergothioneine composition can be used in various forms for human consumption or animal consumption. In some embodiments of any one of the methods or compositions provided herein, the ergothioneine composition can be used in dietary supplements, including human or animal supplements. In some embodiments of any one of the methods or compositions provided herein, the ergothioneine composition can be used in food, including human food, pet food, animal feed (e.g., feed for equine, cattle). In some embodiments of any one of the methods or compositions provided herein, the ergothioneine composition can be used in beverage. In some embodiments of any one of the methods or compositions provided herein, the ergothioneine composition can be used in cosmetic products. In some embodiments of any one of the methods or compositions provided herein, the ergothioneine composition can be used in any dietary products suitable for the methods as described herein. In some embodiments of any one of the methods or compositions provided herein, the ergothioneine composition can be used in any pharmaceutical products, for humans or animals, suitable for the methods as described herein.

In some embodiments of any one of the methods or compositions provided herein, the ergothioneine composition is in the amount of about 0.04 mg/ml, about 0.05 mg/ml, about 0.1 mg/ml, about 0.2 mg/ml, about 0.3 mg/ml, about 0.4 mg/ml, about 0.5 mg/ml, about 0.6 mg/ml, about 0.7 mg/ml, about 0.8 mg/ml, about 0.9 mg/ml, about 1.0 mg/ml, about 1.1 mg/ml, about 1.2 mg/ml, about 1.3 mg/ml, about 1.4 mg/ml, about 1.5 mg/ml, about 1.6 mg/ml, about 1.7 mg/ml, about 1.8 mg/ml, about 1.9 mg/ml, about 2.0 mg/ml, about 2.1 mg/ml, about 2.2 mg/ml, about 2.3 mg/ml, 2.4 mg/ml, about 2.5 mg/ml, about 2.6 mg/ml, about 2.7 mg/ml, 2.8 mg/ml, about 2.9 mg/ml, about 3.0 mg/ml, about 3.1 mg/ml, about 3.2 mg/ml, about 3.3 mg/ml, about 3.4 mg/ml, about 3.5 mg/ml, about 3.6 mg/ml, about 3.7 mg/ml, about 3.8 mg/ml, about 3.9 mg/ml, about 4.0 mg/ml, about 4.1 mg/ml, about 4.2 mg/ml, about 4.3 mg/ml, about 4.4 mg/ml, about 4.5 mg/ml, about 4.6 mg/ml, about 4.7 mg/ml, about 4.8 mg/ml, about 4.9 mg/ml, or about 5.0 mg/ml, inclusive of all ranges and subranges therebetween. In some embodiments of any one of the methods or compositions provided herein, the ergothioneine composition is in the amount of about 0.04 mg/ml to about 0.1 mg/ml, about 0.1 mg/ml to about 0.5 mg/ml, or about 0.5 mg/ml to about 1.0 mg/ml. In some embodiments of any one of the methods or compositions provided herein, the ergothioneine composition is in the amount of about 0.04 mg/ml. In some embodiments of any one of the methods or compositions provided herein, the ergothioneine composition is in the amount of about 0.1 mg/ml. In some embodiments of any one of the methods or compositions provided herein, the ergothioneine composition is in the amount of about 0.3 mg/ml. In some embodiments of any one of the methods or compositions provided herein, the ergothioneine composition is in the amount of about 1 mg/ml.

In some embodiments of any one of the methods or compositions provided herein, the ergothioneine composition is in the amount of about 0.04 mg/g, about 0.05 mg/g, about 0.1 mg/g, about 0.2 mg/g, about 0.3 mg/g, about 0.4 mg/g, about 0.5 mg/g, about 0.6 mg/g, about 0.7 mg/g, about 0.8 mg/g, about 0.9 mg/g, about 1 mg/g, about 1.1 mg/g, about 1.2 mg/g, about 1.3 mg/g, about 1.4 mg/g, about 1.5 mg/g, about 2 mg/g, 2.5 mg/g, 3 mg/g, about 3.5 mg/g, 4 mg/g, 4.5 mg/g, about 5 mg/g, about 6 mg/g, about 7 mg/g, about 8 mg/g, about 9 mg/g, about 10 mg/g, about 11 mg/g, about 12 mg/g, about 13 mg/g, about 14 mg/g, about 15 mg/g, about 20 mg/g, about 25 mg/g, about 30 mg/g, about 35 mg/g, about 40 mg/g, about 45 mg/g, about 50 mg/g, inclusive of all ranges and subranges therebetween. In some embodiments of any one of the methods or compositions provided herein, the ergothioneine composition is in the amount of about 1 mg/g to about 10 mg/g, about 5 mg/g to about 25 mg/g, or about 25 mg/g to about 50 mg/g.

In some embodiments of any one of the methods or compositions provided herein, the ergothioneine composition is in the amount suitable for human subjects in need thereof. In some embodiments of any one of the methods or compositions provided herein, the ergothioneine composition is in the amount suitable for any one of the methods as described herein. For example, the ergothioneine composition is in the amount suitable for improving cell viability and/or maintaining telomere length. For example, the ergothioneine composition is in a therapeutic amount suitable for treating, preventing or reducing the risk of any one of the disorders, diseases or conditions provided herein, such as telomere-related disorders, diseases or other conditions. For example, the ergothioneine composition is in a therapeutic amount suitable for treating, preventing or reducing the risk of premature ageing. For example, the ergothioneine composition is in a therapeutic amount suitable for treating, preventing or reducing the risk of a cognition-related disorder, disease or other condition. For example, the ergothioneine composition is in a therapeutic amount suitable for treating, preventing or reducing the risk of an oxidative stress-related disorder, disease or other condition.

### Methods of Use

Aspects of the present disclosure provide at least in part methods of improving cell viability. In some embodiments, the cell viability is improved by maintaining the average telomere length of cells, such as of a test cell population.

A telomere is a region of repetitive nucleotide sequences at each end of a chromosome, which protects the end of the chromosome from nucleolytic degradation, unnecessary recombination, repair, and interchromosomal fusion. For vertebrates, the sequence of nucleotides in telomeres is TTAGGG. As a normal cellular process, a small portion of telomeric DNA is lost with each cell division. Telomere shortening in humans can induce replicative senescence, which blocks cell division. Progressive shortening of telomeres may lead to senescence, apoptosis, or oncogenic transformation of somatic cells, affecting the health and lifespan of an individual. Therefore, telomere length may serve as a biological clock to determine the lifespan of a cell and an organism. Telomere length is also known as a marker for several diseases such as ageing and cancer. Regarding ageing, it is known that telomere length decreases with age because telomerase activity in adult tissue is not sufficient to prevent telomere shortening, thus compromising cellular viability. Without wishing to be bound by theory, for cancer cells, telomere length is maintained due to the over-expression of telomerase or due to the activation of alternative mechanisms which promote telomerase elongation.

Telomeres are dynamic structures that are impacted both by personal genetics and history as well as numerous environmental influences. Various forms of stress can alter the cellular environment and promote telomere shortening. Oxidative stress can cause telomere shortening in vitro [11]. In vivo, there have been reports of significant correlations between a variety of oxidative stress markers and telomere length and/or shortening, and experimental studies to date seem to also indicate that oxidative stress affects telomere shortening [12]. Additionally, women with higher life stress or psychological stress have been shown to have shorter telomeres that women with low stress [13]. The exact mechanisms linking psychological stress and telomere length are unknown, but without wishing to be bound by theory, it is believed that oxidative stress could be a potential link. Glucocorticoids, which are secreted in response to stress, are known to cause oxidative damage to nerves, and self-reported distress has been linked with greater oxidative DNA damage [14,15]. Oxidative stress has also been implicated in the pathogenesis of a number of chronic diseases which are also associated with shorter telomere length, including cardiovascular disease, diabetes, and cancer [16].

It has been surprisingly found that ergothioneine can exert a protective effect on telomeres under oxidative conditions. Oxidative stress can contribute to telomere shortening and it is demonstrated herein that treatment with ergothioneine can confer beneficial effects by decreasing the rate of telomere shortening and preserving telomere length under oxidative stress conditions. Under oxidative conditions, ergothioneine treatment, for example, significantly increased median telomere length and 20^{th} percentile telomere length, and significantly reduced the percentage of short telomeres (<3 kilobase pairs), with concomitant reductions in telomere shortening rate. Accordingly, in some aspects, the disclosure provides methods of treating, preventing or reducing the risk of a disorder, disease or other condition associated with oxidative stress. In some embodiments, the disclosure provides methods of improving cell viability by protecting telomeres under oxidative conditions (e.g., conditions of oxidative stress). In some embodiments, the cell viability is improved by maintaining the average telomere length of cells, such as of a test cell population (e.g., conditions of oxidative stress).

In some embodiments of any one of the methods provided herein, the average telomere length of cells, such as of a test cell population, contacted or incubated with ergothioneine as described herein can be maintained. In some embodiments of any one of the methods provided herein, the average telomere length of the cells, such as of a test cell population, contacted or incubated with ergothioneine as described herein can be maintained under oxidative conditions (e.g., conditions of oxidative stress). In some embodiments of any one of the methods provided herein, the average telomere length of the cells, such as of a test cell population, contacted or incubated with ergothioneine can be maintained when compared with the average telomere length of control cells, such as of a control cell population, in the absence of the contact or incubation as described herein. For example, the control cells or control cell population can be cells or cell populations that are the same types as the test cells or test cell populations but without the contact or incubation with ergothioneine. Such control cells or cell population may be incubated under standard cell culture conditions as known by a skilled person in the art. In some embodiments, control cells or control cell populations can be different cell types than the test cells or test cell populations. For example, such control cells or control cell populations can be cells that have specifically known and stable telomere lengths. The test cells or test cell populations treated with the ergothioneine as described herein can be compared with the control cells or control cell populations with known and stable telomere length to determine whether the test cells or test cell populations have preserved or maintained length of telomeres. In this instance, the unchanged length or relatively unchanged length of telomeres of the test cells or test cell populations indicate the preservation or maintenance of the telomere length and/or the improvement of cell viability. In some embodiments of any one of the methods provided herein, the “maintaining” or “preserving” the telomere strength refers to the lack of or a minimal indication of the shortening of the telomere length that do not negatively impact cell viability.

In some embodiments of any one of the methods provided herein, the cell viability of the cells, such as test cells or test cell populations, contacted with ergothioneine can be increased by about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%, or about 100%, inclusive of all ranges and subranges therebetween, compared with the cell viability of control cells or control cell populations without contact with ergothioneine.

In some embodiments of any one of the methods provided herein, the cells, such as test cells, can be skin, small intestine, testis, cornea, blood, bone marrow, brain or tumor tissue cells or are of a tissue sample of skin, small intestine, testis, cornea, bone marrow, brain or tumor tissue. In some embodiments of any one of the methods provided herein, the cells, such as the test cells or test cell populations, can be a sample derived from any tissues or organs suitable for the methods as described herein. In some embodiments of any one of the methods provided herein, the control cells can be derived from a Hela 2, HeLa, MCF7, HeLa S3, 293T, L5178Y-S, MEFs BL6 G3 Terc−/−, MEFs BL6 wild type, HeLa 1211, MEFs 129Sv/BL6 wild-type or L5178Y-R cell line. In some embodiments of any one of the methods provided herein, the control cells can be derived from any cells that are suitable for the methods as disclosed herein. In some embodiments of any one of the methods or compositions provided herein, the cells or cell population are from or is a transplantable body part, including whole organs, portions of organs, and tissue, such as heart, lung, heart-lung, kidney, liver, pancreas, intestine, skin, face, hands, leg, penis, bone, uterus, thymus, islets of Langerhans, heart valve, or ovary(ies). In some embodiments of any one of the methods or compositions provided herein, the cells or cell population are from or is a severed limb or portion thereof, including tissue.

In some embodiments, the telomere length can be determined by various methods or assays known in the art. The methods or assays can include but are not limited to a single cell telomeric mapping technique, telomere restriction fragment assay, flow fluorescent in situ hybridization (flow-FISH), hybridization assay, primed in situ labeling (PRIMS), and PCR-based methods such as STELA and quantitative PCR. Regarding the Real-Time PCR assay, the telomere length can be determined by assessing the Telomere-to-Single Copy Gene (T/S) ratio. In some embodiments, the single cell telomeric mapping technique is based on a fluorescent in situ hybridization assay. Methods for the determination of telomere length are described in, and incorporated by reference from, U.S. Pat. No. 8,084,203.

The compositions of the disclosure are also useful in methods for preserving cells, such as may be needed for an organ transplant or for preserving severed limbs or other body parts, including tissue. Thus, aspects of the present disclosure provide methods of preserving an organ for organ transplant or of preserving a severed limb or other body part, including tissue. In some embodiments, the organ for organ transplant is any transplantable organ or body part, including tissue, such as but not limited to, heart, lung, heart-lung, kidney, liver, pancreas, intestine, skin, face, hands, leg, penis, bone, uterus, thymus, islets of Langerhans, heart valve, or ovary(ies). In some embodiments, the methods comprise treating an organ for organ transplant or a severed limb or other body part, including tissue, with an effective amount of a composition comprising ergothioneine.

Aspects of the present disclosure provide methods of treating, preventing or reducing the risk of telomere-related disorders, diseases or other conditions. In some aspects, the telomere-related disorders, diseases or other conditions are cognition-related disorders. Other aspects of the present disclosure provide methods of treating, preventing or reducing the risk of oxidative stress-related disorders, diseases or other conditions. In some embodiments, the methods comprise administering a therapeutically effective amount of composition comprising ergothioneine to a subject in need thereof.

As described herein, “therapeutically effective amount” refers to an amount of the ergothioneine composition that can treat, prevent, or alleviate the symptoms of a disorder, disease or other condition provided herein, such as telomere-related disorders, diseases or other conditions, when administered to a subject in need thereof, and therefore, can achieve at least a therapeutic outcome or effect as understood by a skilled person in the art. Therapeutically effective amounts also refer to an amount of the ergothioneine composition that can be used for any one of the purposes provided in, for example, for maintaining or supporting health, slowing the ageing process, reducing the risk of a disease, disorder or condition, etc.

The dose of the ergothioneine composition required to achieve a particular “therapeutic outcome or effect,” e.g., the mg of purified ergothioneine per ml of the solution for administration, will vary based on several factors including, but not limited to: the route of administration, the body weight of the subject in need thereof, the level of ergothioneine required to achieve a therapeutic effect in various organs or tissues, the specific disease, disorder, or condition being treated, the stability of the ergothioneine composition. etc. One of skill in the art can readily determine a dose range of the ergothioneine composition to treat a subject in need thereof having a particular disease, disorder, or condition based on the aforementioned factors, as well as other factors, as relevant and needed. In some embodiments of any one of the methods or compositions provided herein, the ergothioneine composition can be in any amounts suitable for human subjects in need thereof as described herein. In some embodiments of any one of the methods or compositions provided herein, the ergothioneine composition is in the amount of about 0.04 to about 0.1 mg/ml, about 0.1 to about 0.5 mg/ml, or about 0.5 to about 1.0 mg/ml. In some embodiments of any one of the methods or compositions provided herein, the ergothioneine composition can be in the amount of about 0.04 mg/ml. In some embodiments of any one of the methods or compositions provided herein, the ergothioneine composition can be in the amount of about 0.1 mg/ml. In some embodiments of any one of the methods or compositions provided herein, the ergothioneine composition can be in the amount of about 0.3 mg/ml. In some embodiments of any one of the methods or compositions provided herein, the ergothioneine composition can be in the amount of about 1 mg/ml.

In some embodiments of any one of the methods or compositions provided herein, the ergothioneine composition is in the amount of about 0.04 mg/g, about 0.05 mg/g, about 0.1 mg/g, about 0.2 mg/g, about 0.3 mg/g, about 0.4 mg/g, about 0.5 mg/g, about 0.6 mg/g, about 0.7 mg/g, about 0.8 mg/g, about 0.9 mg/g, about 1 mg/g, about 1.1 mg/g, about 1.2 mg/g, about 1.3 mg/g, about 1.4 mg/g, about 1.5 mg/g, about 2 mg/g, 2.5 mg/g, 3 mg/g, about 3.5 mg/g, 4 mg/g, 4.5 mg/g, about 5 mg/g, about 6 mg/g, about 7 mg/g, about 8 mg/g, about 9 mg/g, about 10 mg/g, about 11 mg/g, about 12 mg/g, about 13 mg/g, about 14 mg/g, about 15 mg/g, about 20 mg/g, about 25 mg/g, about 30 mg/g, about 35 mg/g, about 40 mg/g, about 45 mg/g, about 50 mg/g, inclusive of all ranges and subranges therebetween. In some embodiments of any one of the methods or compositions provided herein, the ergothioneine composition is in the amount of about 1 mg/g to about 10 mg/g, about 5 mg/g to about 25 mg/g, or about 25 mg/g to about 50 mg/g.

In some embodiments of any one of the methods provided herein, the methods comprise administering ergothioneine at a dose of about 5 mg per day, about 6 mg per day, about 7 mg per day, about 8 mg per day, about 9 mg per day, about 10 mg per day, about 11 mg per day, about 12 mg per day, about 13 mg per day, about 14 mg per day, about 15 mg per day, about 16 mg per day, about 17 mg per day, about 18 mg per day, about 19 mg per day, about 20 mg per day, about 21 mg per day, about 22 mg per day, about 23 mg per day, about 24 mg per day, about 25 mg per day, about 26 mg per day, about 27 mg per day, about 28 mg per day, about 29 mg per day, or about 30 mg per day, inclusive of all ranges and subranges therebetween. In some embodiments, ergothioneine is administered at dose of about 5 mg to about 30 mg per day. In some embodiments, ergothioneine is administered at dose of about 5 mg to about 25 mg per day. In some embodiments ergothioneine is administered at a dose of about 5 mg to about 10 mg per day, about 10 mg to about 15 mg per day, about 15 mg to about 20 mg per day, about 20 mg to about 25 mg per day, or about 25 mg to about 30 mg per day. In some embodiments of any one of the methods provided herein, the methods comprise administering ergothioneine at a dose of about 5 mg per day or at least 5 mg per day. In some embodiments of any one of the methods provided herein, the methods comprise administering ergothioneine at a dose of about 10 mg per day or at least 10 mg per day. In some embodiments of any one of the methods provided herein, the methods comprise administering ergothioneine at a dose of about 15 mg per day or at least 15 mg per day. In some embodiments of any one of the methods provided herein, the methods comprise administering ergothioneine at a dose of about 20 mg per day or at least 20 mg per day. In some embodiments of any one of the methods provided herein, the methods comprise administering ergothioneine at a dose of about 25 mg per day or at least 25 mg per day. In some embodiments of any one of the methods provided herein, the methods comprise administering ergothioneine at a dose of about 30 mg per day or at least 30 mg per day. In some embodiments of any one of the methods provided herein, the methods comprise administering ergothioneine at a dose of about 40 mg per day or at least 40 mg per day. In some embodiments of any one of the methods provided herein, the methods comprise administering ergothioneine at a dose of about 50 mg per day or at least 50 mg per day.

Non-limiting examples of telomere-related disorders, diseases or other conditions can include but are not limited to ageing-associated diseases such as atherosclerosis and cardiovascular disease, cancer, arthritis, cataracts, osteoporosis, type 2 diabetes, hypertension and Alzheimer's disease, age-related macular degeneration, benign prostatic hyperplasia, telomere diseases, bone marrow failure, dyskeratosis congenita, acquired aplastic anemia, pulmonary fibrosis, and liver disease. In some embodiments of any one of the methods provided herein, the telomere-related disorder, disease or other condition is or is associated with any one of the foregoing.

In some embodiments of any one of the methods provided herein, the telomere-related disorder, disease or other condition is or is associated with premature ageing.

In some embodiments of any one of the methods provided herein, the telomere-related disorder, disease or other condition is or is associated with a cognition-related disorder, disease or other condition.

In some embodiments of any one of the methods provided herein, the telomere-related disorder, disease or other condition is or is associated with chronic inflammation.

In some embodiments of any one of the methods provided herein, the telomere-related disorder, disease or other condition is caused by or is associated with oxidative stress.

Non-limiting examples of cognition-related disorders, diseases or other conditions include cognitive impairment, mild Cognitive Impairment, frontotemporal dementia, vascular dementia, dementia with Lewy bodies, presenile dementia, senile dementia, Friederich's ataxia, Down's syndrome, Huntington's chorea, hyperkinesia, mania, Tourette's syndrome, Alzheimer's disease, progressive supranuclear palsy, impairment of cognitive functions including attention, orientation, learning disorders, memory (e.g., memory disorders, amnesia, amnesic disorders, transient global amnesia syndrome and age-associated memory impairment) and language function; cognitive impairment as a result of stroke, Huntington's disease, Pick's disease, AIDS-related dementia or other dementia states such as multi-infarct dementia, alcoholic dementia, hypothyroidism-related dementia, and dementia associated to other degenerative disorders such as cerebellar atrophy and amyotropic lateral sclerosis; other acute or sub-acute conditions that may cause cognitive decline such as delirium or depression (pseudodementia states) trauma, head trauma, age related cognitive decline, stroke, neurodegeneration, drug-induced states, neurotoxic agents, age-related cognitive impairment, autism related cognitive impairment, Down's syndrome, cognitive deficit related to psychosis, and post-electroconvulsive treatment related cognitive disorders; cognitive disorders due to drug abuse or drug withdrawal including nicotine, cannabis, amphetamine, cocaine, Attention Deficit Hyperactivity Disorder (ADHD) and dyskinetic disorders such as Parkinson's disease, neuroleptic-induced parkinsonism, and tardive dyskinesias, schizophrenia, schizophreniform diseases, psychotic depression, mania, acute mania, paranoid, hallucinogenic and delusional disorders, personality disorders, obsessive compulsive disorders, schizotypal disorders, delusional disorders, psychosis due to malignancy, metabolic disorder, endocrine disease or narcolepsy, psychosis due to drug abuse or drug withdrawal, bipolar disorders and schizo-affective disorder. In some embodiments, the cognition-related disorder, disease or other condition is Alzheimer's disease, frontotemporal dementia, schizophrenia, Parkinson's disease, Huntington's disease, Lewy body disease, vascular dementia, traumatic brain injury (TBI), Parkinson's disease, prion disease, dementia, amnesia, or delirium.

Non-limiting examples of disorders, diseases or other conditions associated with oxidative stress include cancer, neurodegenerative disease (e.g., Parkinson's disease, Alzheimer's disease, multiple sclerosis and amyolotrophic lateral sclerosis), eye disorders cardiovascular disease, atherosclerosis, sickle cell disease, thrombotic thrombocytopenic purpura, sepsis, cystic fibrosis, chronic fatigue syndrome, kidney disease, diabetes, acute respiratory distress syndrome, gout, arthritis, and other inflammatory diseases. Oxidative stress is involved in several age-related conditions (e.g., cardiovascular diseases, chronic obstructive pulmonary disease, chronic kidney disease, neurodegenerative diseases, and cancer), including sarcopenia and frailty. Oxidative stress is also involved in viral infections (e.g., HCV infection, HIV infection, etc.). In some embodiments, the viral infection is a respiratory viral infection (e.g., an infection caused by influenza virus, influenza (IV), human respiratory syncytial (HRSV), human rhinovirus (HRV), human metapneumovirus (HMPV), parainfluenza, and adenovirus and coronavirus (CoV) (e.g., SARS-CoV, MERS, and SARS-CoV-2)). In some embodiments, the viral infection is COVID-19, caused by SARS-CoV-2. Without wishing to be bound by theory, it is believed that ergothioneine will improve COVID-19 symptoms by exerting a protective effect on telomeres under oxidative conditions. As an example, it is believed that ergothioneine can treat or prevent inflammation and/or cytokine storms, protect against acute respiratory distress syndrome, mitigate oxidative stress, lung damage, and other long-term conditions, etc. associated with COVID-19.

Non-limiting examples of inflammatory diseases or disorders include Alzheimer's, arthritis, asthma, atherosclerosis, Crohn's disease, colitis, cystic fibrosis, dermatitis, diverticulitis, hepatitis, irritable bowel syndrome (IBS), lupus erythematous, muscular dystrophy, nephritis, Parkinson's, rheumatoid arthritis, shingles and ulcerative colitis. Inflammatory diseases also include, for example, stroke, cardiovascular disease, chronic obstructive pulmonary disease (COPD), bronchiectasis, chronic cholecystitis, tuberculosis, Hashimoto's thyroiditis, kidney fibrosis, sepsis, sarcoidosis, silicosis and other pneumoconioses.

In some embodiments of any one of the methods provided herein, the disease, disorder or condition is preeclampsia, overhydrated hereditary stomatocytosis, lung injury, fertility (e.g., sperm motility, embryo development, embryo quality), cataracts.

In some embodiments of any one of the methods provided herein, the treating, preventing or reducing the risk of the telomere-related, oxidative-stress-related, inflammation-related or cognition-related disorders, diseases or other conditions comprises maintaining the average telomere length of cells in a target environment in a subject in need thereof. In some embodiments of any one of the methods provided herein, maintaining the average telomere length of cells in a target environment in a subject in need thereof improves cell viability and/or stability.

In some embodiments of any one of the methods or compositions provided herein, the ergothioneine is L-ergothioneine. In some embodiments of any one of the methods or compositions provided herein, ergothioneine is ErgoActive®.

In some embodiments of any one of the methods or compositions provided herein, the ergothioneine is purified. In some embodiments of any one of the methods or compositions provided herein, the purified ergothioneine has purity of about or at least 98% or 99% or more. In some embodiments of any one of the methods or compositions provided herein, the purified ergothioneine can have any purity as described herein.

In some embodiments of any one of the methods or compositions provided herein, the ergothioneine is in the amount of any one of the amounts provided herein. In some embodiments of any one of the methods or compositions provided herein, the ergothioneine can be in any amounts that are suitable for the methods as described herein.

In some embodiments of any one of the methods or compositions provided herein, the ergothioneine composition can be in a free-flowing powder form. In some embodiments of any one of the methods or compositions provided herein, the ergothioneine composition can be in any forms as described herein.

### Pharmaceutical Compositions

Aspects of the present disclosure provide, in some embodiments, a composition comprising a therapeutically effective amount of ergothioneine, such as L-ergothioneine, such as a natural form of purified L-ergothioneine. The composition can be a pharmaceutical composition. In some embodiments of any one of the methods or compositions provided herein, the pharmaceutical composition can include one or more pharmaceutically acceptable carriers. Purified ergothioneine can have purity of about 98%, 99% or more, or any other purity level that is suitable for the methods and the formulating of the composition, such as the pharmaceutical composition, as disclosed herein.

Suitable carriers may be readily selected by one of skill in the art in view of the types of antioxidants or dietary supplements. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present disclosure.

In some embodiments of any one of the methods or compositions provided herein, the pharmaceutical composition comprising a therapeutically effective amount of ergothioneine can comprise other pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin. In some embodiments of any one of the methods or compositions provided herein, the composition, such as the pharmaceutical composition, comprising a therapeutically effective amount of ergothioneine can comprise flavoring agents or sweeteners.

Methods described herein comprise administering the composition, such as the pharmaceutical composition, comprising a therapeutically effective amount of ergothioneine in sufficient amounts to treat or prevent any one of the disorders, diseases or other conditions provided hereinwithout undue adverse effects. Routes of administration are known to those of skill in the art and may be combined, if desired.

As described herein, the dose or amounts of ergothioneine required to achieve a particular “therapeutic outcome or effect” will vary based on several factors. For example, a therapeutically effective amount of ergothioneine is an amount sufficient to alleviate one or more symptoms of any one of the diseases, disorders or conditions provided herein. The therapeutically effective amount would depend on factors such as the age, gender, weight, health of the subject, and the relevant tissues. In some embodiments of any one of the methods or compositions provided herein, a therapeutically effective amount of ergothioneine is any one of the amounts provided herein in, for example, mg/ml, mg/g or mg/d.

In an example, a therapeutically effective amount of ergothioneine can be generally in the range of from about 0.1 mg/ml to 100 mg/ml, about 1 mg/ml to 90 mg/ml, about 0.04 mg/ml to about 1 mg/ml, etc. In some embodiments, a therapeutically effective amount of ergothioneine can be 0.04 mg/ml. In some embodiments, a therapeutically effective amount of ergothioneine can be 0.1 mg/ml. In some embodiments, a therapeutically effective amount of ergothioneine can be 0.3 mg/ml. In some embodiments, a therapeutically effective amount of ergothioneine can be 1 mg/ml.

In some embodiments, a therapeutically effective amount of ergothioneine is about 5 mg to about 50 mg per day. In some embodiments, a therapeutically effective amount of ergothioneine is about 5 mg to about 25 mg per day. In some embodiments, a therapeutically effective amount of ergothioneine is about 5 mg to about 10 mg per day, about 10 mg to about 15 mg per day, about 15 mg to about 20 mg per day, about 20 mg to about 25 mg per day, or about 25 mg to 30 mg per day. In some embodiments, a therapeutically effective amount of ergothioneine is about 5 mg per day or at least 5 mg per day. In some embodiments, a therapeutically effective amount of ergothioneine is about 10 mg per day or at least 10 mg per day. In some embodiments, a therapeutically effective amount of ergothioneine is about 15 mg per day or at least 15 mg per day. In some embodiments, a therapeutically effective amount of ergothioneine is about 20 mg per day or at least 20 mg per day. In some embodiments, a therapeutically effective amount of ergothioneine is about 25 mg per day or at least 25 mg per day. In some embodiments, a therapeutically effective amount of ergothioneine is about 30 mg per day or at least 30 mg per day.

In some embodiments, ergothioneine is administered to a subject as frequently as needed to treat or prevent a disorder, disease or other condition. In some embodiments, ergothioneine is administered to a subject as frequently as needed to improve cell viability and/or maintain telomere length. In some embodiments, ergothioneine is administered daily. In some embodiments, ergothioneine is administered at least once a week (1, 2, 3, 4, 5, or 6 times a week), weekly, fortnightly, or monthly. In some embodiments, ergothioneine is administered to a subject for as long as needed to improve cell viability and/or maintain telomere length. In some embodiments, ergothioneine is administered to a subject for 1 week, 2 weeks, 3 weeks, 4 week, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, or a year, inclusive of all ranges and subranges therebetween.

Formulation of pharmaceutically-acceptable excipients and carrier solutions is known to those of skill in the art, as is the development of suitable dosing and treatment regimens for using the particular compositions described herein. The amount of active compound in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such formulations, and as such, a variety of dosages and treatment regimens may be desirable.

The forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringeability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

The ergothioneine composition can be formulated or prepared in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is known in the art. Supplementary active ingredients can also be incorporated into the compositions. The phrase “pharmaceutically-acceptable” refers to molecular entities and compositions that do not produce an allergic or similar untoward reaction when administered to a host.

### Food/Beverage and Supplement Products

In some embodiments of any one of the methods or compositions provided herein, the ergothioneine composition can be formulated or prepared as a food supplement for oral consumption. In some embodiments of any one of the methods or compositions provided herein, the ergothioneine composition can be formulated or prepared for cosmetic product application. In some embodiments of any one of the methods or compositions provided herein, the ergothioneine composition can be formulated or prepared for dietary products, such as food products, medical foods, etc. In some embodiments of any one of the methods or compositions provided herein, the ergothioneine composition can be formulated or prepared for beverages.

Food compositions according to the invention include any preparations or compositions which are suitable for consumption and are used for nutrition or enjoyment purposes. They are generally products which are intended to be eaten by humans or animals and introduced into the body through the mouth, to remain there for a certain time and then either be eaten (e.g., ready-to-eat foodstuffs or feeds, see also herein below) or removed (e.g. chewing gums). Such products include any substances or products which in the processed, partially processed or unprocessed state are to be ingested by humans or animals. They also include substances which are added to orally consumable products during their manufacture, preparation or treatment and which are intended to be introduced into the human or animal oral cavity.

The food compositions according to the invention also include substances which in the unchanged, treated or prepared state are to be swallowed by a human or animal and then digested; in this respect, the orally consumable products according to the invention also include casings, coatings or other encapsulations which are to be swallowed at the same time or which may be expected to be swallowed. The expression “food composition” covers ready-to-eat foodstuffs, beverages and feeds, that is to say foodstuffs, beverages or feeds that are already complete in terms of the substances that are important for the taste. The expressions “ready-to-eat foodstuff” and “ready-to-eat feed” also include drinks as well as solid or semi-solid ready-to-eat foodstuffs or feeds. Examples which may be mentioned are frozen products, which must be thawed and heated to eating temperature before they are eaten. Products such as yoghurt or ice-cream as well as chewing gums or hard caramels are also included among the ready-to-eat foodstuffs or feeds of the current invention.

Non-limiting examples of food and beverage products include carbonated soft drinks, ready to drink beverages, energy drinks, isotonic drinks, low-calorie drinks, zero-calorie drinks, sports drinks, teas, fruit and vegetable juices, juice drinks, dairy drinks, yoghurt drinks, alcohol beverages, powdered beverages, bakery products, cookies, biscuits, baking mixes, cereals, confectioneries, candies, toffees, chewing gum, dairy products, flavored milk, yoghurts, flavored yoghurts, cultured milk, soy sauce and other soy base products, salad dressings, mayonnaise, vinegar, frozen-desserts, meat products, fish-meat products, bottled and canned foods, tabletop sweeteners, fruits and vegetables.

The compositions can be used “as-is” or in combination with sweeteners, flavors and food ingredients. Dry compositions, such as powders, granules or tablets are stable indefinitely when stored under dry conditions at room temperature. Compositions in the form of aqueous solutions are stable indefinitely when frozen. If a preservative such as benzoic acid or its salts, sulphur dioxide or sodium meta-bisulphite is added to such a composition, it may be stored almost indefinitely at room temperature.

Non-limiting examples of flavors include lemon, orange, fruity, banana, grape, pear, pineapple, bitter almond, cola, cinnamon, sugar, cotton candy, vanilla flavors.

Non-limiting examples of other food ingredients include flavors, acidulants, organic and amino acids, coloring agents, bulking agents, modified starches, gums, texturizers, preservatives, antioxidants, emulsifiers, stabilizers, thickeners, gelling agents.

### EXAMPLES

### Example 1: Microbial Ergothioneine Biosynthesis

Ergothioneine or L-ergothioneine (e.g., ErgoActive®) is produced in an engineered microbial system. Briefly, E. coli are transformed with the pConB7A vector and the pConA5K vectors encoding EgtB, EgtC, EgtD and EgtE. To co-express the four genes (EgtB, C, D, E) in E. coli system, the transformants are grown in the LB media containing 100 mg/L ampicillin and 50 mg/L kanamycin at 37° C. until reaching an OD6oo˜0.6. Expression is induced by the addition of 0.2-0.5 mM of isopropyl β-D-1-thiogalactopyranoside (IPTG) and the culture is further grown at either 30° C. or 37° C. for 16-24 hours. Cells are harvested by centrifugation and the supernatant and cell pellet are collected separately. The supernatant is centrifuged at 16,000×g for 5 min for HPLC analysis. The pellet is resuspended in 1 ml of 50% methanol and sonicated for 1 minute (3×20 sec). After centrifuging at 16,000×g for 5 minutes, 5 μl of sample is analyzed by HPLC, as described below. E. coli that are transformed with the empty vector are treated in the same manner and analyzed by HPLC. A sample obtained from IPTG-induced E. coli containing the EgtB, EgtC, EgtD, EgtE genes is spiked with 20 mg/L ergothioneine and analyzed by HPLC.

Samples are analyzed using a Dionex UPLC Ultimate 3000 (Sunnyvale, Calif.). The compounds are separated on an Atlantis HILIC Silica column (particle size 3.0 m, diameter×length=2.1×100 mm; Waters) and detected at 264 nm. The mobile phase consisted of 0.1% formic acid in water (A) and 0.1% formic acid in acetonitrile (B). The program of a gradient is 95% B at 1 min, 40% B at 8 minutes, 95% B at 8.1 minutes, stop at 1 1 min. The flow rate is 0.6 ml/minute and the inject volume is 5 μl.

In addition, the retention time of the ergothioneine from the E. coli strain containing EgtB, EgtC, EgtD and EgtE overlaps with the retention time of the ergothioneine standard. In addition to retention time, the UV-spectrum of the ergothioneine peak also matches the ergothioneine standard. Other methods of microbial ergothioneine biosynthesis is described in, and incorporated by reference from PCT/US2015/027977.

### Example 2: Determination of Telomere Length

To determine the effects on the changes of telomere length, several methods can be used, some of which are described, and are incorporated by reference from U.S. Pat. No. 8,084,203. As an example, for telomere length analyses, samples from mouse tail and back skin, small intestine, cornea, testis and brain are harvested and fixed o/n in neutral buffered formalin at 4° C., dehydrated through graded alcohols and xylene, and embedded in paraffin. Prior to embedding, dissected skin is cut parallel to the spine in order to obtain longitudinal hair follicle sections. The intestinal tract is flushed with PBS and rolled up in a compact circle using longitudinally oriented jejunal sections for analysis. For cornea and testis analyses, whole eyes and testis are cut in half prior to dehydration. Finally, brain is coronal-dissected to harvest the rostral hippocampus. In all cases, 5″ M sections are used for QFISH and immunostaining analyses. As an example of the use of Flow-FISH telomere length measurements in K15-EGFP mice, freshly isolated keratinocyte suspensions (EGFP+ and EGFP−) from K15-EGFP mice are fixed in methanol/acetic acid (3:1), permeabilized with methanol 100% and washed in PBS. Cells are blocked in BSA 10% PBS for 15 m at RT and incubated with BD Living Colours AV Monoclonal Antibody JL-8 (Becton Dickinson, San Jose, Calif.) at 1:250 dilution for 30 min at RT. After two washes in Tween20-PBS cells are blocked in BSA 10% PBS for 15 m at RT and incubated with goat antibody to mouse conjugated with Alexa 647 at 1:500 dilution (Molecular Probes, Invitrogen) 30 m at RT. After two washes in Tween20-PBS cells are fixed in formaldehyde 0.5% PBS for 5 m and washed twice in PBS. Then telomere flow-FISH is performed as described (Rufer et al., 1998) using a FITC labeled PNA-tel probe and Propidium Iodide (PI, Sigma) to counterstain DNA, and analyzed in a FACScanto cytometer (BD Biosciences). Cells with adequate size and complexity as determined by forward scatter and side scatter channels, are gated for G0/G1 phase using the PI signal acquired in FL2 channel. Their labeling for Alexa 467 is acquired in FL4 channel and is used to identify GFP positive and negative cell populations. The telomere fluorescence as FITC signal is acquired in FL1 for both cell populations. To compensate for the contribution of cellular autofluorescence, fluorescence values of negative control cells (i.e., cells hybridized in the absence of the FITC PNA-tel probe) are subtracted from every sample. L5178Y-R and L5178Y-S cell lines with known telomere length of 10.2 and 79.7 kb (McIlracth et al., 2001), respectively, are processed in parallel and used to convert fluorescence values into kb. Negative controls for each fluorochrome and acquisition settings are established with unstained or single stained cell populations.

### Example 3: Effect of Ergothioneine on Telomere Length and Rate of Shortening

### Materials and Methods

### Cell Culture and Reagents

Primary cultures of human neonatal dermal fibroblasts (ATCC® PCS-201-010™) were established under standard culture conditions. Cells were seeded at 3×10³ cells/cm² and were grown and maintained in Fibroblast Medium (Innoprot).

Ergothioneine at ≥98% was produced by Blue California through a proprietary fermentation process utilizing a modified strain of Escherichia coli K12, which is nonpathogenic and nontoxigenic and contains genes encoding proteins that convert histidine to ergothioneine.

### MIT Assay

The MTT assay is a colorimetric assay that uses a yellow tetrazolium salt (3-(4,5-dimethylthiazol-2-yl)-2,5-diphenyltetrazolium bromide, or MTT) to measure metabolic activity of cells as a proxy for cell viability. MTT reduction rate is an indicator of the functional integrity of the mitochondria and as a result, of cellular viability [17, 18]. To determine the cytotoxicity of ergothioneine on primary neonatal fibroblast cells, the cells were seeded in 96 well plates (Nunc) at 0.35×10⁴ cells/plate and 0.5×10⁴ cells/plate for 72 hours and 1-week treatment. After 24 hours from seeding, the cells were washed once with phosphate-buffered saline (PBS) and treated with the respective compounds in cell culture media. Ergothioneine was dissolved in dimethyl sulfoxide (DMSO). Eight serial dilutions were done (1, 0.5, 0.25, 0.125, 0.0625, 0.0313, 0.0156, 0.00078 mg/ml) to evaluate the compound. Each condition was tested in triplicates with and without 10 μM H₂O₂. For positive and negative controls, 8 mM methyl methane sulfonate (MMS) and DMSO 100% were used, respectively. Following compound addition, the plates were incubated for 72 hours and one week. The media with the compound was changed every two days. After the treatment period, the cells were washed twice with PBS and media was replaced with MTT reagent at 0.5 mg/ml in Dulbecco's Modified Eagle Medium (DMEM) without phenol red. The plates were gently shaken and incubated for 4 hours. After the incubation, the medium was removed and replaced by DMSO. The plates were gently shaken to solubilize the formazan crystals. Absorbance was measured using an Envision multiplate reader at a wavelength of 570 nm.

The percentage of cell death was calculated using the following equation:

Cell death (%)=(ODₛₐₘₚₗₑ−OD_{PC}/OD_{NC}−OD_{PC})×100

Where OD_{NC}, is the optical density of the negative control and OD_{PC} is the optical density of the positive control.

### Proliferative Analysis

Primary cultures of neonatal human fibroblast cells were established. Cells were seeded at 5×10³ cells/cm²), in Fibroblast Medium (Innoprot). Media was renewed every 2 to 3 days and cells were passaged at sub-confluence (70-80%) every 7 days. Cells were expanded during eight weeks under standard and oxidative (10 μM H₂O₂) cell culture condition as described previously. Different concentrations of ergothioneine (0.04, 0.3, 0.1, and 1.0 mg/ml) or vehicle control were added to the treated or control cells, respectively. Treatment dilutions were prepared fresh in each passage with fresh media. Cell growth was monitored for each condition by counting cell numbers at each passage using a Countess™ cell counter (Invitrogen). Population doubling (PD) was calculated with the formula PD=3.322(Log(Cf)−Log (Ci))+X (where, Cf: Final concentration; Ci: initial concentration; X: PD last passage). One PD is equivalent to one round of cell replication.

Each treatment was tested in triplicate and in different plates. Each plate contained a control sample. Frozen vials were obtained at different time points for analysis of relative telomerase activity and telomere length.

### Relative Telomerase Activity

The quantitative polymerase chain reaction (PCR)-based telomeric repeat amplification protocol (Q-TRAP) is a sensitive and accurate PCR-based assay that enables the measurement of telomerase activity. Cellular pellets from neonatal fibroblast cells were lysed using the CHAPS (3-cholamidopropyl dimethylammonio 1-propanesulfonate) lysis buffer for protein extraction [19]. Samples were stored at 4° C. and were used within 24 hours. Protein quantification was performed for each sample using Biorad protein assay (Bio-Rad Kit 50000002). A minimum of 0.3 mg/ml protein concentration was required to proceed with the analysis of the samples. Telomerase protein extracts are then incubated at 27° C. for 30 min to allow the telomerase in the protein extracts to elongate the TS primer by adding TTAGGG repeat sequences. Following the enzymatic reaction, telomerase extension products are then amplified and quantified by real-time qPCR using SYBR Green (a green fluorescent cyanine dye) [19-21]. PCR was initiated at 95° C. for 10 min, followed by a 40-cycle amplification (95° C. for 15 s, 60° C. for 60 s) and a melt curve stage step. Reactions were monitored and analyzed with QuantStudio 5 (Applied Biosystems). Telomerase activity in cell lines or samples is calculated based on the threshold cycle (Ct). All samples were run in triplicate.

The telomerase-positive standard dilution series is plotted against the telomere protein concentration (r²>0.9) as a standard curve of Ct values. The standard curve is generated by graphing threshold cycles (Ct values) of HeLa cell line standards against log of 1000, 333, 111, 37.03, 12.34, 4.11, 1.37 and 0.45 ng of protein (whole cell extract).

### Telomere Length Measurement Using Telomere Analysis Technology (TAT®)

All telomere length measurements were performed using Life Length's proprietary Telomere Analysis Technology (TAT®). TAT® measures telomere length using a high-throughput (HT) Q-FISH (quantitative fluorescent in situ hybridization) technique as previously described [22]. This method is based on a quantitative fluorescence in-situ hybridization method modified for measuring individual chromosomes of cells in interphase. In brief, telomeres are hybridized with a fluorescent Peptide Nucleic Acid probe (PNA) that recognizes three telomere repeats (sequence: Alexa488-OO-CCCTAACCCTAACCCTAA, purchased from Panagene). The images of the nuclei and telomeres are captured by a high-content screen system. The intensity of the fluorescent signal from the telomeric PNA probes that hybridize to a given telomere is proportional to the length of that telomere. The intensities of fluorescence are translated to base pairs (bp) through a standard regression curve which is generated using control cell lines with known telomere length. TAT® not only measures telomere length in absolute base pair units, it also provides an assessment of the distribution, 50th and 20th percentiles of telomere length and the percent of short telomeres, allowing for a more comprehensive analysis of each sample. All samples are run in quintuplicate.

### Statistical Analysis

Results obtained from each treatment and at each time point were compared with the appropriate control group using Student's t test (independent samples t-test) [23].

### Results

### Cell Viability

All concentrations of ergothioneine tested (0.04, 0.1, 0.3, and 1.0 mg/ml) produced cell death values <20% indicating no significant toxic effects. Evaluation by morphological analysis through optical microscopy visualization revealed that ergothioneine at concentrations of up to 1.0 mg/ml did not produce any deleterious effects.

### Proliferative Analysis

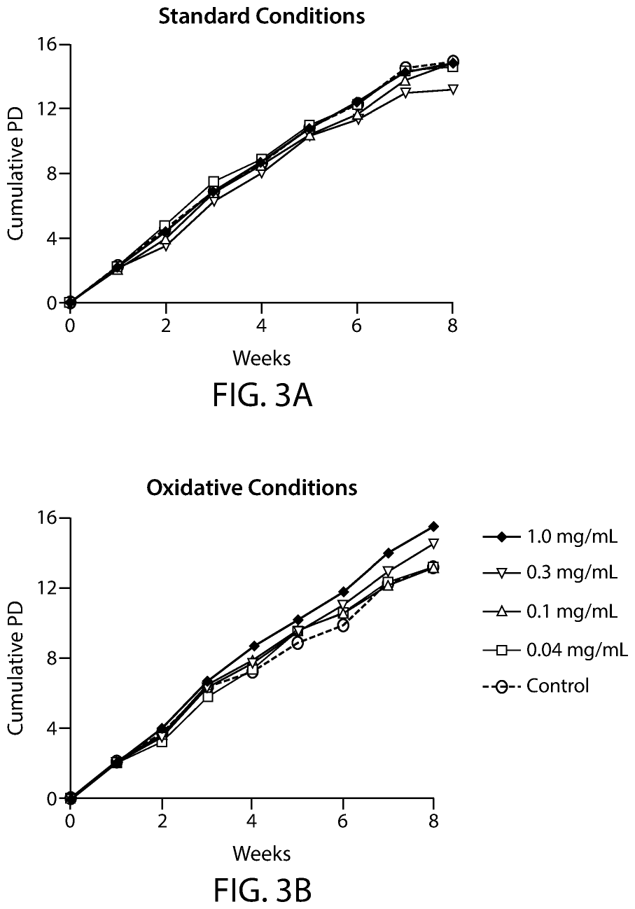

Under standard culture conditions, no differences were identified in the proliferative capacity between control and treated fibroblasts (FIG. 3A). Under oxidative culture conditions, cells treated with ergothioneine at 1.0 or 0.3 mg/ml showed an increase in their proliferative capacity compared to the control group after Week 4 (FIG. 3B).

### Telomerase Activity

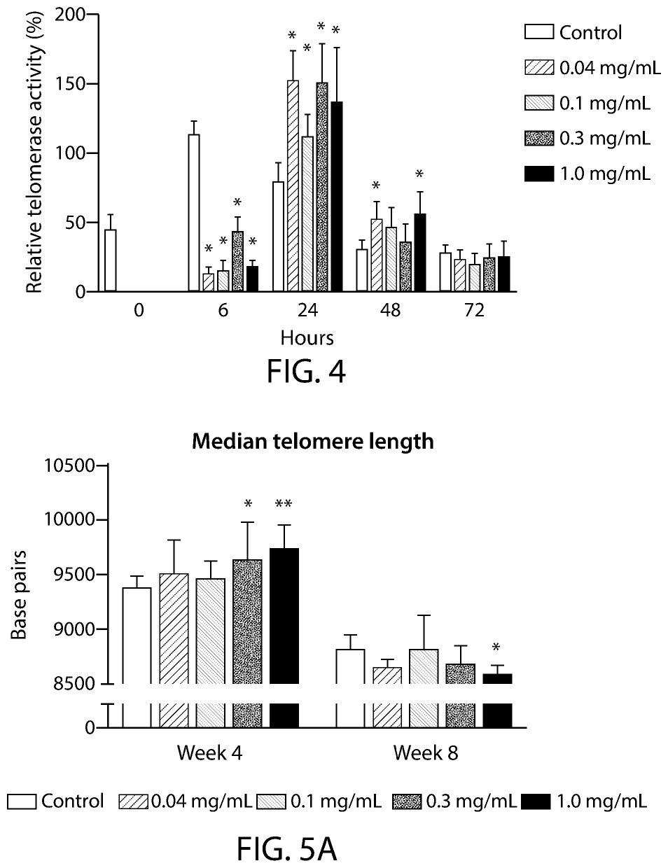

Telomerase activity was measured in human fibroblasts over a 72-hour period (FIG. 4). At 6 hours, the telomerase activity in ergothioneine-treated cells at all dose levels was significantly lower than in control cells. At 24 hours, telomerase activity was 50-90% higher in ergothioneine-treated cells at all dose levels compared with control cells (p<0.05 for all dose levels). At 48 hours, telomerase activity was still elevated compared with control, though this effect was only statistically significant for 0.04 mg/ml and 1.0 mg/ml ergothioneine. At 72 hours, telomerase activity was not significantly different between the treatment groups and control.

### Telomere Length

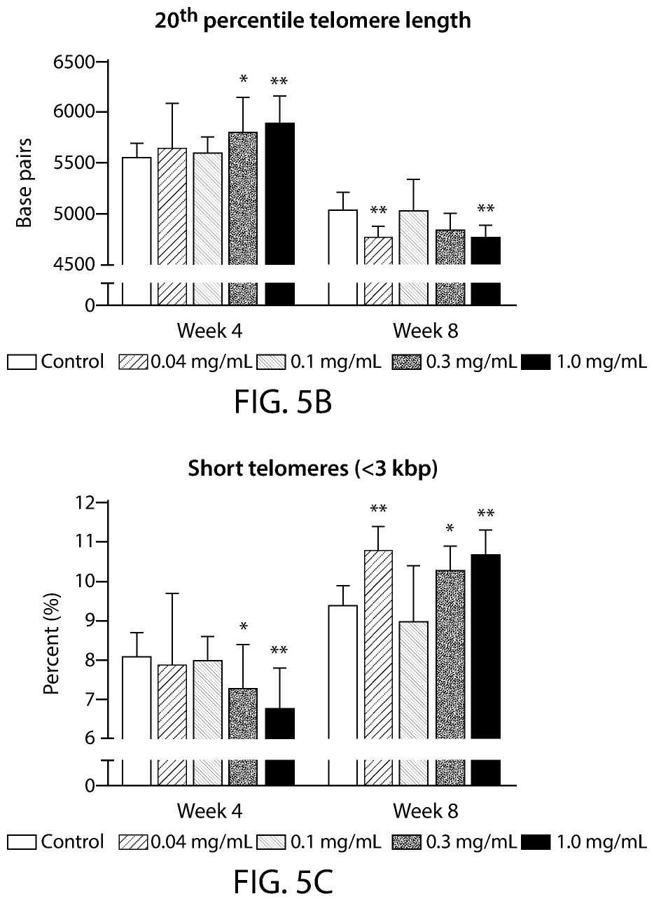

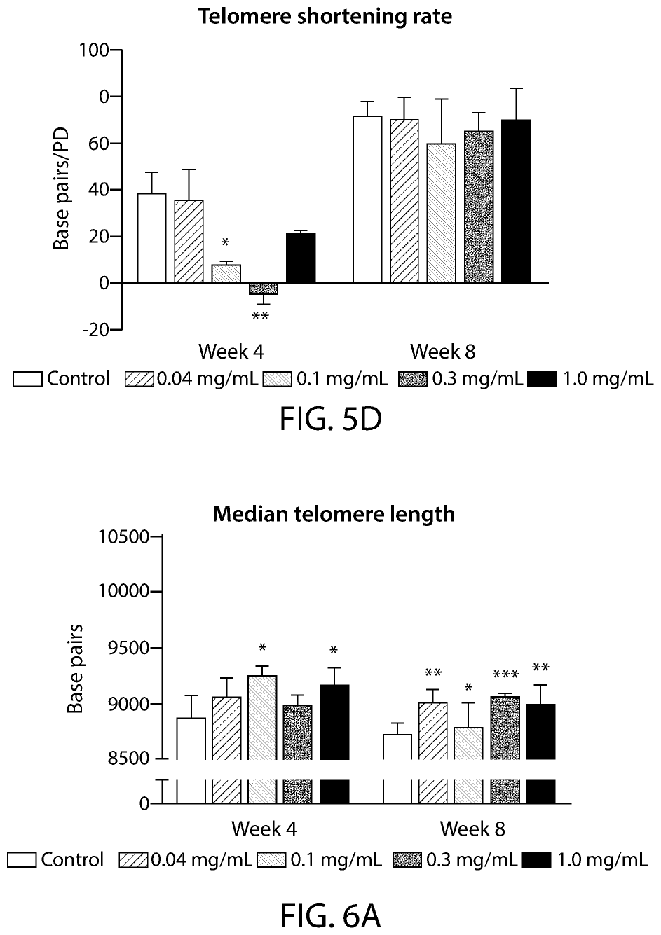

Telomere length was evaluated under standard and oxidative (10 μM H₂O₂) cell culture conditions over an 8-week period. At Week 4, cells in standard conditions treated with 0.3 mg/ml or 1.0 mg/ml ergothioneine had greater median telomere length and 20^{th} percentile telomere length compared with control (FIGS. 5A and 5B). Median telomere length was 9378±107 base pairs (bp) for control cells, 9637±340 bp for 0.3 mg/ml ergothioneine, and 9745±206 bp for 1.0 mg/ml ergothioneine. Cells treated with 0.3 or 1.0 mg/ml ergothioneine also had a significantly lower percentage of short telomeres (<3 kbp or <3000 bp) at Week 4 versus control (FIG. 5C). The proportion of short telomeres (<3 kbp) was 8.1±0.6% for control, 7.3±1.1% for 0.3 mg/ml ergothioneine, and 6.8±1.0% for 1.0 mg/ml ergothioneine. Telomere shortening rates were assessed and normalized by the number of population doublings (cell replications) to assess the specific effect of ergothioneine. Telomere shortening rate at Week 4 was significantly lower for cells treated with 0.1 or 0.3 mg/ml ergothioneine compared with control (FIG. 5D), and there was a trend for decreased telomere shortening rate with 1.0 mg/ml ergothioneine (p=0.09). At Week 8, all the protective effects previously seen at Week 4 were no longer present (FIG. 5).

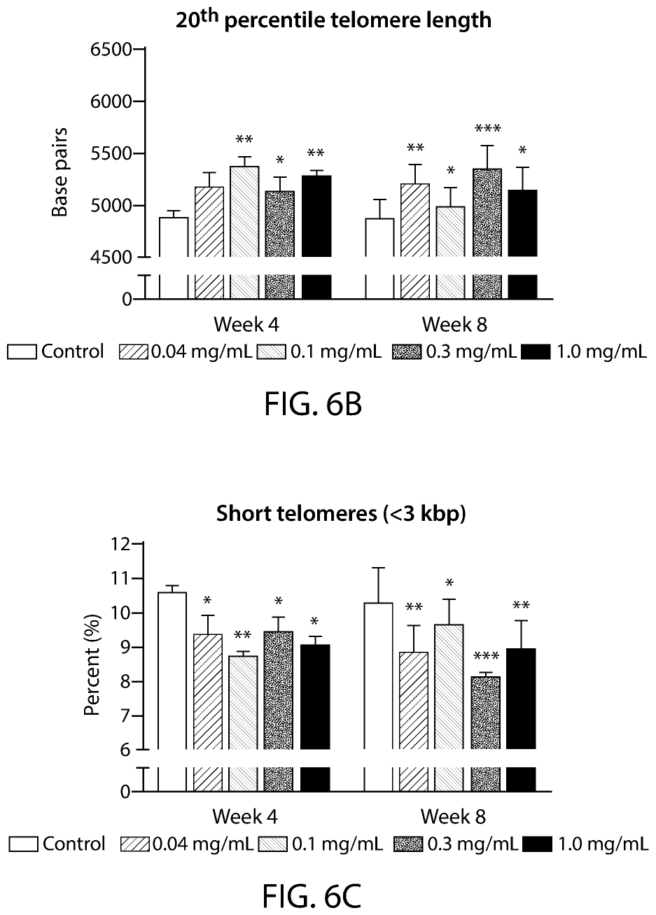

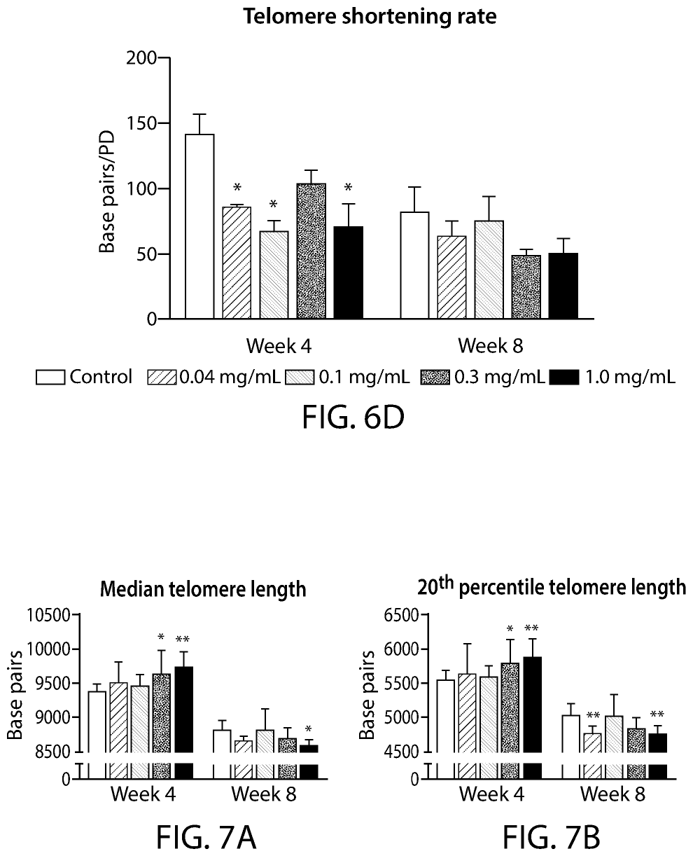

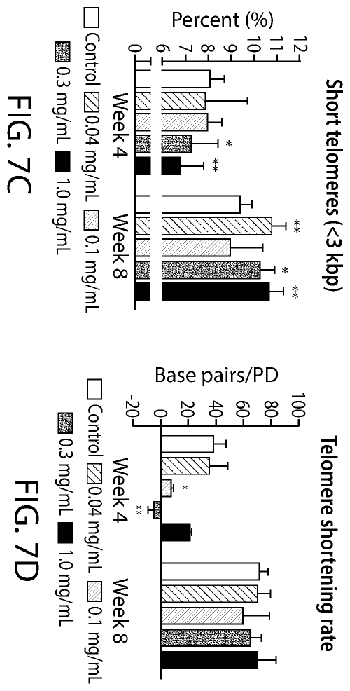

Under oxidative conditions, ergothioneine appeared to have greater protective effects. At Week 4, cells treated with 0.1 or 1.0 mg/ml ergothioneine had a higher median telomere length versus control (p<0.05, FIG. 6A), and at Week 8 treatment with all concentrations of ergothioneine significantly increased median telomere length versus control (FIG. 6A). At Week 8 under oxidative conditions, median telomere length was, 9015±112 bp for 0.04 mg/ml, 8794±212 bp for 0.1 mg/ml, 9068±24 for 0.3 mg/ml, and 8997±171 bp for 1.0 mg/ml ergothioneine compared with 8725±94 bp for control (p<0.05 for all). Results for 20^{th} percentile telomere length were similar with treatment, with 0.1, 0.3, or 1.0 mg/ml ergothioneine resulting in greater telomere length (p<0.05) at Week 4. These effects carried over to Week 8, when all concentrations of ergothioneine produced significant increases in 20th percentile telomere length compared with control (FIG. 6B). All concentrations of ergothioneine reduced the percentage of short telomeres (<3 kbp) both at Week 4 and Week 8 versus control (FIG. 6C). At Week 4, all concentrations of ergothioneine reduced telomere shortening rate by 27-52%, and this was statistically significant for 0.04 mg/ml, 0.1 mg/ml, and 1.0 mg/ml (FIG. 6D). At Week 8, telomere shortening rate appeared to be lower in ergothioneine-treated groups, but these effects did not reach statistical significance.

### Discussion

The results demonstrate that ergothioneine can exert a protective effect on telomeres especially under oxidative conditions. Testing was conducted under both standard and oxidative conditions. Over the course of 8 weeks under oxidative conditions, treatment with ergothioneine resulted in longer median telomere length, longer 20^{th} percentile telomere length, a lower percentage of short telomeres (<3 kbp), and concomitant reductions in telomere shortening rate. Because telomere shortening rate is normalized using the number of population doublings, the observed reductions in telomere shortening rate indicate these reductions were due to ergothioneine treatment rather than a difference in the number of cell divisions. These beneficial effects are further supported by observed transient increases in relative telomerase activity with ergothioneine treatment. The effects did not appear to be dose-dependent.

One of the concerns in working with cultured cells is that they are known to have a limited capacity to divide, after which they become senescent. Previous work has shown that proliferation of cells in culture is initially robust but then declines over time, and after approximately 50 replicative cycles the cells become senescent, a number known as Hayflick's limit [24]. Over the course of 8 weeks, the cells in our experiments underwent 13-16 replicative cycles, which is well below Hayflick's limit. Cells could also become senescent if telomeres become critically short, though there is no standard threshold for what is considered critically short. Median telomere length in our study ranged from approximately 8.5 to 9.8 kbp which is well above what would be considered short, and only 7-11% of telomeres were less than 3 kbp. Both the proliferation data and the telomere length data indicate that the cells used this study were likely not senescent.

The concentrations of ergothioneine used in this study (17 μM to 4.4 mM) are within physiological ranges that have been observed in tissues and blood. Previous studies have reported high ergothioneine concentrations of 100 μM to 2 mM in erythrocytes, bone marrow, liver, kidney, seminal fluid, and the lens and cornea of the eye [25]. In humans, supplementation with ergothioneine for 7 days resulted in concentrations of approximately 3 μM in plasma and 30-40 μM in whole blood [26]. Thus, in our study, ergothioneine at physiological concentrations demonstrated beneficial effects by decreasing the rate of telomere shortening and preserving telomere length under oxidative stress conditions. Overall, our data support a potential role for ergothioneine in healthy ageing.

### Example 4: Use of Ergothioneine to Treat Cognition-Related Disorders, Diseases or Other Conditions

An effective amount of a pharmaceutical composition comprising ergothioneine is administered to an older adult patient suffering from a cognition-related disorder or disease in a 16-week study. The primary outcome variable measured is the change from baseline to week 16 in the composite memory standard score from the CNS-VS test battery. Secondary outcome variables include one or more of: changes from baseline to weeks 4 and 8 in the CNS-VS composite memory standard score; changes from baseline to weeks 4, 8, and 16 in the following CNS-VS domains: neurocognition index, verbal memory, visual memory, psychomotor speed, reaction time, complex attention, cognitive flexibility, processing speed, executive function, simple attention, and motor speed; changes from baseline to weeks 4, 8, and 16 in the LSEQ; changes from baseline to weeks 4, 8, and 16 in the POMS Questionnaire; changes from baseline to weeks 4, 8, and 16 in hs-CRP; changes from baseline to weeks 4, 8, and 16 in ergothioneine levels; change from baseline to week 16 in telomere length. Plasma and/or serum samples are archived at visits 1b-4 for analyses of non-genetic markers of inflammation.

Ergothioneine can be effective in maintaining telomere length, improving cell viability, and thus improving the symptoms associated with the cognition-related disorder or disease.

### Example 5: Use of Ergothioneine to Treat Oxidative Stress Related Viral Infections

The purpose of this study is to determine whether ergothioneine is safe and effective to treat patients with oxidative stress related viral infections (e.g., HCV infection, HIV infection, etc.). In some embodiments, the viral infection is a respiratory viral infection (e.g., an infection caused by influenza virus, influenza (IV), human respiratory syncytial (HRSV), human rhinovirus (HRV), human metapneumovirus (HMPV), parainfluenza, and adenovirus and coronavirus (CoV) (e.g., SARS-CoV, MERS, and SARS-CoV-2)). In some embodiments, the viral infection is COVID-19, caused by SARS-CoV-2. Without wishing to be bound by theory, it is believed that ergothioneine will improve symptoms by exerting a protective effect on telomeres under oxidative conditions. As an example, it is believed that ergothioneine can treat or prevent inflammation and/or cytokine storms, protect against acute respiratory distress syndrome, mitigate oxidative stress, lung damage, and other long-term conditions, etc.

### Example 6: Use of Ergothioneine to Treat COVID-19

The purpose of this study is to determine whether ergothioneine is safe and effective to treat patients with COVID-19. An effective amount of a pharmaceutical composition comprising ergothioneine is administered to a patient suffering from COVID-19 to treat the disease. Ergothioneine can exert a protective effect on telomeres especially under oxidative conditions prevalent in COVID-19 patients, improving cell viability, and thus improving the symptoms of COVID-19.

### REFERENCES

1. Hernández et al., U.S. Pat. No. 8,084,203B2, Methods for the determination of telomere length in a semi-automatic manner of every single cell in a immobilized cell population.
2. Han et al., PCT/US2015/027977, Microbial ergothioneine biosynthesis.
3. Kerley et al., The Potential Therapeutic Effects of Ergothioneine in Pre-eclampsia. Free Radical Biology and Medicine, DOI: 10.1016/j.freeradbiomed.2017.12.030.
4. Richard-Greenblattet et al. Regulation of Ergothioneine Biosynthesis and Its Effect on Mycobacterium tuberculosis Growth and Infectivity. (2015) J Bio Chem 290 (38): 23064-23076.
5. Van der Hoek et al. Engineering the Yeast Saccharomyces cerevisiae for the Production of L-(+)-Ergothioneine (2019) Front. Bioeng. Biotechnol. https://doi.org/10.3389/fbioe.2019.00262.
6. Harley et al., Telomeres shorten during ageing of human fibroblasts (1990) Nature, 345(6274):458-60.
7. Blasco et al., Telomere shortening and tumor formation by mouse cells lacking telomerase RNA (1997) Cell, 91(1):25-34.
8. Kim et al., Specific association of human telomerase activity with immortal cells and cancer (1994) Science, 266(5193):2011-5.
9. Bryan et al., Evidence for an alternative mechanism for maintaining telomere length in human tumors and tumor-derived cell lines (1997) Nat Med, 3(11):1271-4.
10. Calado et al., Telomere Diseases (2009), N Engl J Med. 10; 361(24): 2353-2365.
11. von Zglinicki, T. Oxidative stress shortens telomeres. Trends Biochem Sci 2002, 27, 339-344, doi:10.1016/s0968-0004(02)02110-2.
12. Reichert, S.; Stier, A. Does oxidative stress shorten telomeres in vivo? A review. Biol Lett 2017, 13, doi:10.1098/rsbl.2017.0463.
13. Epel, E. S.; Blackburn, E. H.; Lin, J.; Dhabhar, F. S.; Adler, N. E.; Morrow, J. D.; Cawthon, R. M. Accelerated telomere shortening in response to life stress. Proc Natl Acad Sci USA 2004, 101, 17312-17315, doi:10.1073/pnas.0407162101.
14. McIntosh, L. J.; Sapolsky, R. M. Glucocorticoids may enhance oxygen radical-mediated neurotoxicity. Neurotoxicology 1996, 17, 873-882.
15. Irie, M.; Asami, S.; Ikeda, M.; Kasai, H. Depressive state relates to female oxidative DNA damage via neutrophil activation. Biochem Biophys Res Commun 2003, 311, 1014-1018, doi:10.1016/j.bbrc.2003.10.105.
16. Liguori, I.; Russo, G.; Curcio, F.; Bulli, G.; Aran, L.; Della-Morte, D.; Gargiulo, G.; Testa, G.; Cacciatore, F.; Bonaduce, D., et al. Oxidative stress, aging, and diseases. Clin Interv Aging 2018, 13, 757-772, doi:10.2147/CIA.S158513.
17. Verhulst, C.; Coiffard, C.; Coiffard, L. J.; Rivalland, P.; De Roeck-Holtzhauer, Y. In vitro correlation between two colorimetric assays and the pyruvic acid consumption by fibroblasts cultured to determine the sodium laurylsulfate cytotoxicity. J Pharmacol Toxicol Methods 1998, 39, 143-146, doi:10.1016/s1056-8719(98)00016-1.
18. Liu, Y.; Nair, M. G. An efficient and economical MTT assay for determining the antioxidant activity of plant natural product extracts and pure compounds. J Nat Prod 2010, 73, 1193-1195, doi:10.1021/np1000945.
19. Hou, M.; Xu, D.; Bjorkholm, M.; Gruber, A. Real-time quantitative telomeric repeat amplification protocol assay for the detection of telomerase activity. Clin Chem 2001, 47, 519-524.
20. Herbert, B. S.; Hochreiter, A. E.; Wright, W. E.; Shay, J. W. Nonradioactive detection of telomerase activity using the telomeric repeat amplification protocol. Nat Protoc 2006, 1, 1583-1590, doi:10.1038/nprot.2006.239.
21. Wege, H.; Chui, M. S.; Le, H. T.; Tran, J. M.; Zern, M. A. SYBR Green real-time telomeric repeat amplification protocol for the rapid quantification of telomerase activity. Nucleic Acids Res 2003, 31, E3-3, doi:10.1093/nar/gng003.
22. de Pedro, N.; Diez, M.; Garcia, I.; Garcia, J.; Otero, L.; Fernandez, L.; Garcia, B.; Gonzalez, R.; Rincon, S.; Perez, D., et al. Analytical Validation of Telomere Analysis Technology® for the High-Throughput Analysis of Multiple Telomere-Associated Variables. Biol Proced Online 2020, 22, 2, doi:10.1186/s12575-019-0115-z.
23. Mishra, P.; Pandey, C. M.; Singh, U.; Keshri, A.; Sabaretnam, M. Selection of appropriate statistical methods for data analysis. Ann Card Anaesth 2019, 22, 297-301, doi:10.4103/aca.ACA_248_18.
24. Campisi, J.; d'Adda di Fagagna, F. Cellular senescence: when bad things happen to good cells. Nat Rev Mol Cell Biol 2007, 8, 729-740, doi:10.1038/nrm2233.
25. Cheah, I. K.; Halliwell, B. Ergothioneine; antioxidant potential, physiological function and role in disease. Biochim Biophys Acta 2012, 1822, 784-793, doi:10.1016/j.bbadis.2011.09.017.
26. Cheah, I. K.; Tang, R. M.; Yew, T. S.; Lim, K. H.; Halliwell, B. Administration of Pure Ergothioneine to Healthy Human Subjects: Uptake, Metabolism, and Effects on Biomarkers of Oxidative Damage and Inflammation. Antioxid Redox Signal 2017, 26, 193-206, doi:10.1089/ars.2016.6778.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A method of improving cell viability or maintaining telomere length comprising contacting or incubating cells, such as a test cell population, with a composition comprising ergothioneine (e.g., a natural form of purified ergothioneine), optionally, wherein the ergothioneine has purity of at least or about 98%.

2. The method of claim 1, wherein the ergothioneine comprises L-ergothioneine.

3. The method of any one of claim 1-2, wherein the cell viability is improved by maintaining the average telomere length of the cells such as of the test cell population.

4. The method of any one of claims 1-3, wherein the telomere length is determined by a single cell telomeric mapping technique.

5. The method of claim 4, wherein the single cell telomeric mapping technique is based on a fluorescent in situ hybridization assay.

6. The method of any one of the preceding claims, wherein the composition is in a free-flowing powder form.

7. The method of any one of the preceding claims, wherein the average telomere length of the cells, such as of the test cell population, is maintained when compared with the average telomere length of control cells, such as a control cell population, in the absence of contact or the incubation with the ergothioneine.

8. The method of any one of the preceding claims, wherein the cells, such as the test cells or test cell population, is a tissue sample selected from the group consisting of skin, small intestine, testis, cornea, blood, bone marrow, brain and tumor tissues.

9. The method of any one of the preceding claims, wherein the control cells are of an immortalized cell line selected from the group consisting of Hela 2, HeLa, MCF7, HeLa S3, 293T, L5178Y-S, MEFs BL6 G3 Terc−/−, MEFs BL6 wild type, HeLa 1211, MEFs 129Sv/BL6 wild-type and L5178Y-R.

10. The method of any one of the preceding claims, wherein the ergothioneine contacted with or incubated with the cells, such as in the test cell population, is in the amount of about 0.04-1.0 mg/ml.

11. The method of any one of the preceding claims, wherein the ergothioneine contacted with or incubated with the cells, such as in the test cell population, is in the amount of about 0.04 mg/ml, about 0.1 mg/ml, about 0.3 mg/ml, or about 1 mg/ml.

12. A method of treating or preventing telomere-related disorders, diseases or other conditions comprising administering a therapeutically effective amount of composition comprising ergothioneine (e.g., a natural form of purified ergothioneine) to a subject in need thereof, optionally, wherein the ergothioneine has purity of at least or about 98%.

13. The method of claim 12, wherein the ergothioneine comprises L-ergothioneine.

14. The method of claim 12 or 13, wherein the composition is in a free-flowing powder form.

15. The method of any one of claims 12-14, wherein the treating or preventing the telomere-related disorders, diseases or other conditions comprises maintaining the average telomere length of cells in a target environment in the subject in need thereof.

16. The method of any one of claims 12-15, wherein the telomere-related disorders, diseases or other conditions are associated with premature ageing.

17. The method of any one of claims 12-15, wherein the telomere-related disorders, diseases or other conditions are associated with chronic inflammation.

18. The method of any one of claims 12-15, wherein the telomere-related disorders, diseases or other conditions are caused by or associated with oxidative stress.

19. A method of treating or preventing a cognition-related disorder, disease or other condition comprising administering a therapeutically effective amount of composition comprising ergothioneine (e.g., a natural form of purified ergothioneine) to a subject in need thereof, optionally, wherein the ergothioneine has purity of at least or about 98%.

20. The method of claim 19, wherein the ergothioneine comprises L-ergothioneine.

21. The method of claim 19 or 20, wherein the composition is in a free-flowing powder form.

22. The method of any one of claims 19-21, wherein the treating or preventing the cognition-related disorders, diseases or other conditions comprises maintaining the average telomere length of cells in a target environment in the subject in need thereof.

23. The method of any one of claims 19-22, wherein the cognition-related disorder, disease or other condition is associated with premature ageing.

24. The method of any one of claims 19-22, wherein the cognition-related disorder, disease or other condition is associated with chronic inflammation.

25. The method of any one of claims 19-22, wherein the cognition-related disorder, disease or other condition is Alzheimer's disease, frontotemporal dementia, schizophrenia, Huntington's disease, Lewy body disease, vascular dementia, traumatic brain injury (TBI), Parkinson's disease, prion disease, dementia, amnesia, or delirium.

26. A method of treating or preventing an oxidative stress-related disorder, disease or other condition, comprising administering a therapeutically effective amount of composition comprising ergothioneine (e.g., a natural form of purified ergothioneine) to a subject in need thereof, optionally, wherein the ergothioneine has purity of at least or about 98%.

27. The method of claim 26, wherein the ergothioneine comprises L-ergothioneine.

28. The method of claim 26 or 27, wherein the composition is in a free-flowing powder form.

29. The method of any one of claims 26-28, wherein the treating or preventing the oxidative stress-related disorders, diseases or other conditions comprises maintaining the average telomere length of cells in a target environment in the subject in need thereof.

30. The method of any one of claims 26-29, wherein the oxidative stress-related disorder, disease or other condition is associated with telomere shortening.

31. The method of any one of claims 26-30, wherein the oxidative stress-related disorder, disease or other condition is associated with premature ageing.

32. The method of any one of claims 26-30, wherein the oxidative stress-related disorder, disease or other condition is associated with chronic inflammation.

33. The method of any one of claims 26-30, wherein the oxidative stress-related disorder, disease or other condition is a viral infection, cardiovascular disease, diabetes, diabetes-related complications, cancer, gout, or arthritis.

34. The method of claim 33, wherein the oxidative-stress related disorder, disease, or other condition is a respiratory viral infection.

35. The method of claim 33 or 34, wherein the viral infection is COVID-19.

36. The method of any one of claims 12-35, wherein the ergothioneine is administered at a dose of about 5 mg to about 25 mg per day.

37. The method of claim 36, wherein the ergothioneine is administered at a dose of about 5 mg per day or about 25 mg per day.

38. A composition comprising ergothioneine (e.g., a natural form of purified L-ergothioneine) and, optionally, one or more pharmaceutical acceptable carriers, and further optionally, wherein the ergothioneine has a purity of at least or about 98%.

39. The composition of claim 38, wherein the ergothioneine comprises L-ergothioneine.

40. The composition of claim 38 or 39, wherein the composition is in a free-flowing powder form.

41. The composition of any one of claims 38-40, wherein the ergothioneine is in the amount of about 0.04-1.0 mg/ml.

42. The composition of claim 41, wherein the ergothioneine is in the amount of about 0.04-0.1 mg/ml, about 0.1-0.5 mg/ml, or about 0.5-1.0 mg/ml.

43. The composition of claim 41, wherein the ergothioneine is in the amount of about 0.04 mg/ml, about 0.1 mg/ml, about 0.3 mg/ml, or about 1 mg/ml.

44. A pharmaceutical formulation comprising a therapeutically effective amount of a composition of any one of claims 38-43 and one or more pharmaceutically acceptable carriers.


SEQUENCE LISTING (1 sequence)

SEQ ID NO: 1 - DNA, 18 bp, Artificial Sequence
  Alexa488
ccctaaccctaaccctaa